(19) United States
(12) Patent Application Publication
(10) Pub. No.: US 2024/0173310 A1
(43) Pub. Date: May 30, 2024

(54) PHARMACEUTICAL DOSAGE FORMS COMPRISING (4S)-24-CHLORO-4-ETHYL-73-FLUORO-35-METHOXY-32,5-DIOXO-14-(TRIFLUOROMETHYL)-32H-6-AZA-3(4,1)-PYRIDINA-1(1)-[1,2,3]TRIAZOLA-2(1,2),7(1)-DIBENZENAHEPTAPHANE-74-CARBOXAMIDE

(71) Applicant: Bayer Aktiengesellschaft, LEVERKUSEN (DE)

(72) Inventors: Susanne Weisweg-Merkel, Solingen (DE); Peter Serno, Bergisch Gladbach (DE)

(21) Appl. No.: 18/549,187

(22) PCT Filed: Mar. 4, 2022
(86) PCT No.: PCT/EP2022/055519

§ 371 Date: Sep. 6, 2023

(30) Foreign Application Priority Data
Mar. 9, 2021 (EP) 21161493.8

(51) Int. Cl. A61K 31/4439; A61K 9/00; A61K 9/20; A61K 9/16

(52) U.S. Cl. CPC A61K 31/4439; A61K 9/0053; A61K 9/2027; A61K 9/1682

(57) ABSTRACT

The present invention relates to amorphous solid dispersions (ASD) and solid pharmaceutical dosage forms for oral administration comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzena-heptaphane-7⁴-carboxamide (active ingredient (I)), characterized in that the active ingredient (I) is immediately released from the amorphous solid dispersions (ASD) and the solid pharmaceutical dosage forms for oral administration, and also methods for the preparation thereof, use thereof as medicaments, and also use thereof for the treatment and/or prophylaxis of diseases, in particular cardiovascular disorders, preferably thrombotic or thromboembolic disorders, and oedemas, and also ophthalmic disorders.

The present invention relates to amorphous solid dispersions (ASD) and solid pharmaceutical dosage forms for oral administration comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzena-heptaphane-7⁴-carboxamide (active ingredient (I)), characterized in that the active ingredient (I) is immediately released from the amorphous solid dispersions (ASD) and the solid pharmaceutical dosage forms for oral administration, and also methods for the preparation thereof, use thereof as medicaments, and also use thereof for the treatment and/or prophylaxis of diseases, in particular cardiovascular disorders, preferably thrombotic or thromboembolic disorders, and oedemas, and also ophthalmic disorders.

The active ingredient (I), (4S)-2⁴-chloro-4-ethyl-7³-fluoro-3⁵-methoxy-3²,5-dioxo-14-(trifluoromethyl)-3²H-6-aza-3(4,1)-pyridina-1(1)-[1,2,3]triazola-2(1,2),7(1)-dibenzenaheptaphane-7⁴-carboxamide, also named as 4-({(2S)-2-[4-{5-chloro-2-[4-(trifluoromethyl)-1H-1,2,3-triazol-1-yl]phenyl}-5-methoxy-2-oxopyridin-1(2H)-yl]butanoyl}amino)-2-fluorobenzamide, is known from WO 2017/005725 and has the following formula:

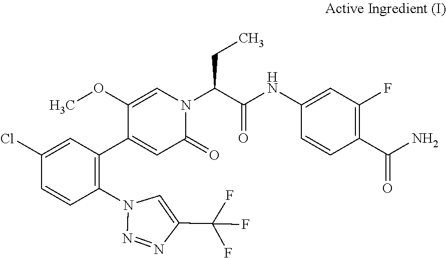 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]

The active ingredient (I) acts as a factor XIa inhibitor and, owing to this specific mechanism of action, is, after oral administration, useful in the treatment and/or prophylaxis of disorders, preferably thrombotic or thromboembolic disorders and/or thrombotic or thromboembolic complications, in particular cardiovascular disorders including coronary artery disease, angina pectoris, myocardial infarction or stent thrombosis, as well as disorders in the cerebrovascular arteries and other disorders, leading to transitory ischaemic attacks (TIA), ischemic strokes including cardioembolic as well as non-cardioembolic strokes, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

To develop a solid pharmaceutical dosage form for oral administration, active ingredient (I), (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide, is used as amorphous form or as crystalline modification I or as a mixture of amorphous form and crystalline modification I.

In cases of diseases which require treatment over a lengthy period, or for the long-term prophylaxis of diseases, it is desirable to keep the frequency of intake of medicaments as low as possible and the tablet size as small as possible. This is not only more convenient for the patient, it also increases the reliability of treatment by reducing the disadvantages of irregular intake (improvement in compliance). In order to increase compliance, particularly in older patients, the tablets should be as small as possible, i.e. have a high concentration of active ingredient, particularly with regard to the higher dosage strengths.

During course of development, it was found that the active ingredient (I) is available in at least two solid state forms, the amorphous form and the crystalline modification I. Also, during course of development, it was found that the relative bioavailability in rats decreases down to 11% when the crystalline modification I of active ingredient (I) is administered. Also, the dissolution behavior of the oral solid dosage forms is inferior when the oral solid dosage forms such as tablets are manufactured by standard methods known to those skilled in the art, containing the crystalline modification I of active ingredient (I). Additionally, the active ingredient (I) is available in two enantiomeric forms, of which one form is ineffective in-vivo.

The aim of the development was, therefore, to provide solid pharmaceutical dosage forms for oral administration comprising (4S)-2⁴-chloro-4-ethyl-7³-fluoro-3⁵-methoxy-3²,5-dioxo-1⁴-(trifluoro-methyl)-3²H-6-aza-3(4,1)-pyridina-1(1)-[1,2,3]triazola-2(1,2),7(1)-dibenzenaheptaphane-7⁴-carboxamide (active ingredient (I)), where the oral solid dosage form shows a superior dissolution behaviour and a good bioavailability of the active ingredient (I). In addition, the enantiomeric purity of the active ingredient (I) should be safeguarded not only in the solid pharmaceutical dosage form but also during the manufacturing process of the same. A high drug-load shall be achieved (>20% active ingredient (I) per dosage form) to ensure a minimal tablet size. Furthermore, the amorphous form of the active ingredient (I) shall be stable in the solid pharmaceutical dosage forms during long-term storage.

Surprisingly, a solid pharmaceutical dosage form comprising an amorphous solid dispersion (ASD) in which the active ingredient (I) is present in an amorphous form shows a superior dissolution behaviour and a good bioavailability. In addition, the enantiomeric purity of the active ingredient (I) could be safeguarded not only in the amorphous solid dispersion (ASD) but also in the solid pharmaceutical dosage form and during the manufacturing process of the same using wet granulation, whereas at the same time a high drug-load (>20% active ingredient (I) per dosage form) could be achieved. The amorphous form of the active ingredient (I) is stabilized by excipients in the amorphous solid dispersion (ASD). As active ingredient (I) is not stabilized in the standard IR tablet, dissolution and bioavailability cannot be achieved with standard formulation approaches which do not inhibit crystallisation of amorphous active ingredient (I). Crystallization of the active ingredient (I) results in a lower dissolution rate and a lower bioavailability of the active ingredient (I).

Furthermore, a manufacturing process with a combination of certain excipients and a combination of certain solvents allows to start the manufacturing process with the active ingredient (I) in amorphous form and/or in crystalline modification I and results in solid pharmaceutical dosage forms for oral administration comprising (4S)-2⁴-chloro-4-ethyl-7³-fluoro-3⁵-methoxy-3²,5-dioxo-1⁴-(trifluoro-methyl)-3²H-6-aza-3(4,1)-pyridina-1(1)-[1,2,3]triazola-2(1,2),7(1)-dibenzenaheptaphane-7⁴-carboxamide (active ingredient (I)), where the amorphous form of the active ingredient (I) is stabilized in an amorphous solid dispersion (ASD).

In the context of the present invention, the term amorphous solid dispersion (ASD) is used whereas in the literature some authors use the term solid solution which has the same meaning as solid dispersion in the context of the present invention.

In the following, the different types of solid dispersions (solid solutions, glass solutions, glass suspensions, amorphous precipitations in a crystalline carrier, eutectics or monotecics, compound or complex formation and combinations thereof) are collectively referred to as solid dispersion.

In the context of the present invention, the term crystallization means crystallization as the active ingredient was not crystalline before and/or recrystallization as the active ingredient was crystalline, was then transferred in the amorphous form, stabilized in the amorphous form and does thereafter crystalize again.

A “matrix” according to the present invention are polymeric excipients, non-polymeric excipients and combinations thereof, capable of dissolving or dispersing the active ingredient (I). In the context of the present invention the “matrix” consists of the combination of the “solid dispersion base” and the “carrier” used during the manufacturing process of the amorphous solid dispersion (ASD). Thus the “matrix” becomes an integral part of the amorphous solid dispersion (ASD). In the prior art some authors use the term carrier instead of matrix or instead of a matrix agent.

Amorphous solid dispersions (ASD) and manufacturing processes thereof as such are known.

C. Leuner and J. Dressman, Eur. J. Pharm. Biopharm., 50 (2000) 47 to 60, divide the methods for preparing solid dispersions roughly in hot melt method and solvent methods. According to C. Leuner and J. Dressman application of hot melt extrusion to the production of solid dispersions is regarded as method for choice for preparing solid dispersion. The authors favor hot melt extrusion as non-solvent method because small variations in the conditions used to remove the solvent can lead to quite large changes in product performance.

J. Breitenbach, Eur. J. Pharm. and Biopharm., 54 (2002) 107-117, classifies the most relevant technologies for the manufacture of solid dispersions in hot spin mixing, embeddings by means of spray-drying, co-evaporation, co-precipitation, freeze-drying and roll-mixing or co-milling. In general, methods based on solvent evaporation known are e.g. freeze drying, spray drying, vacuum drying, layering of powders, granulates or pellets and fluidized bed granulation. J. Breitenbach states that solid dispersions show different and mostly unpredictable behavior with regard to e.g. chemical and physical stability.

R. J. Chokshi et al., J. Pharm. Sci., 97(6) (2008) 2286-2298 and G. P. Andrews et al., J. Pharm. Pharmacol. 62 (2010) 1580-1590, state that solid dispersions are inherently unstable. Solid-dispersion approaches to drug dissolution enhancement involve the generation of a glass solution in which the drug is present in a metastable amorphous state possessing high internal energy and specific volume. This results in a system with a tendency for crystallization during storage.

The choice of the manufacturing process and the parameters applied for as well as the identification of suitable excipients to avoid crystallization is also not obvious for a person skilled in art. According to N. Shah et al., 2014, page 130 (FIG. 4.2), a variety of potential excipients can be considered for composing amorphous solid dispersions. Not all of these excipients are equally suited to prepare an amorphous solid dispersion.

G. Van den Mooter, Drug Discovery Today: Technologies, Vol. 9 (2) (2012) e79-e85 describes the use and manufacturing of amorphous solid dispersions and mentions that beside enormous research efforts both in academia and pharmaceutical industry very few products relying on solid dispersion technology reached the market. Problems of physical stability during shelf life are recognized as the primary reason for this discrepancy.

So called multicomponent solid dispersions in which one or more drugs are dispersed in a (carrier) matrix composed by at least two compounds possessing properties capable to modify or to enhance the drug delivery system in terms of drug release, drug permeability, thermodynamic stability and thus affecting bioavailability are described in L. M. De Mohac, et al., Journal of Drug Delivery Science and Technology, 57 (2020) 101750.

K. Six et al., J. Pharm. Sic., 93 (2004) 124-131 investigated solid dispersions of itraconazole in a combination of two polymers (PVPVA64, a copolymer of N-vinylpyrrolidone and vinyl acetate, and Eudragit E100, an amino methacrylate copolymer) and found that the stability and dissolution rate of solid dispersions comprising combined polymers were superior to those which could be achieved by using one single polymer only.

However, in Y. Huang et al., Acta Pharmaceutica Sinica B, 4(1) (2014) 18-25 is emphasized that the intermolecular drug-polymer interaction has always been/is still the determining factor in the design and performance of solid dispersions. Stability of solid dispersions will also be influenced by moisture absorption during storage.

For instance, polyvinylpyrrolidone (PVP) is known for its absorption of moisture, and polymers that are resistant to water absorption, such as hydroxyproyl methyl cellulose acetate succinate (HPMCAS) have become the first choice for the preparation of stable solid dispersions, mentioned in D. T. Friesen et al., Mol. Pharm., 5(6) (2008) 1003-1019.

In dependence of the drug-carrier combination the release characteristics of the solid solution vary and therefore they are one of the main influences on the performance of a solid dispersion. C. Leuner and J. Dressman, Eur. J. Pharm. Biopharm., 50 (2000) 47 to 60 emphasized that the drug-carrier ratio has to be identified very thoroughly to find an optimal ratio for the formulation. Release rates might become slower with increased carrier amount. Depending on the carrier or carrier mixture incorporated even gel-formation can occur which results in fall-off of the release rate. Therefore, it is neither obvious for a person skilled in the art which excipient(s) to use nor which manufacturing process to apply for preparation of an amorphous solid dispersions containing active ingredient (I).

Furthermore, WO2020/210629 describes an amorphous solid dispersion (ASD) comprising the Factor XIa inhibitor (9R,13S)-13-{4-[5-chloro-2-(4-chloro-1H-1,2,3-triazol-1-yl)phenyl]-6-oxo-1,6-dihydropyrimidin-1-yl}-3-(difluoromethyl)-9-methyl-3,4,7,15-tetraazatricyclo[12.3.1.0^{2,6}]octa-deca-1(18),2(6),4,14,16-pentaen-8-one. The compound is formulated as an amorphous solid dispersion in pharmaceutically relevant polymers such as hydroxyproyl methyl cellulose acetate succinate (HPMCAS) by spray drying and the amorphous solid dispersion has a drug-load of less than 20% active ingredient (I) per dosage form.

As mentioned above the most frequent challenges to prepare an amorphous solid dispersion (ASD) are the physical stability of the amorphous solid dispersion, the type and the amount of matrix, the ratio of drug and matrix needed to facilitate the required increase in release rate and the selection of an appropriate manufacturing and its scale-up for safe-guarding physical and chemical stability of the amorphous solid dispersion and the incorporated drug.

It was surprisingly found that an amorphous solid dispersion (ASD) according to the present invention comprising active ingredient (I) with superior dissolution behaviour and a good bioavailability of the active ingredient (I) as well as safeguard of the enantiomeric purity of the active ingredient (I) is dependent on a) the matrix used, b) the solvent used in the manufacturing process and c) the manufacturing process. The methods and excipients chosen for the present invention are in some aspects in contrast to the methods and excipients known by the person skilled in the art and which are known as common to prepare an amorphous solid dispersion (ASD).

a) The Matrix

It was also surprisingly found that the amorphous form of active ingredient (I) comprised in an amorphous solid dispersion according to the present invention could be stabilized by applying one single polymer as matrix only, which is also in contrast to prior art (K. Six et al., J. Pharm. Sic., 93 (2004) 124-131 and L. M. De Mohac, et al., Journal of Drug Delivery Science and Technology, 57 (2020) 101750) which favors multicomponent systems which apply at least two polymers as matrix.

It was additionally surprising that with using polyvinylpyrrolidone (PVP) as matrix polymer an amorphous solid dispersion comprising active ingredient (I) according to the present invention could be manufactured in which the amorphous form of active ingredient (I) is stabilized not only during the manufacturing process but also during long-term or open storage which is in contrast to D. T. Friesen at al. where PVP is described as inferior due to its water absorption behavior. Surprisingly, hydroxyproyl methyl cellulose acetate succinate (HPMCAS) did not lead to the desired dissolution criteria (release of at least 85% of active ingredient (I) into the release medium after an investigation period of 30 minutes) and may not stabilize the active ingredient (I) in its amorphous form which is also in contrast to D. T. Friesen et al. where HPMCAS is given preference to PVP.

The advantage of the present invention is that the amorphous form of active ingredient (I) can be stabilized in an amorphous solid dispersion (ASD) by applying polyvinylpyrrolidone (PVP) as single polymer in the matrix only, resulting in a solid pharmaceutical dosage form with high drug-load (>20% active ingredient (I) per dosage form), which allows a small tablet size.

B) The Solvent Used in the Manufacturing Process

Surprisingly, the combination of the solvents ethanol and acetone let to a more than 5-fold improved solubility compared to the solubility if the pure ethanol is used and let to an approximately 2-fold improved solubility compared to the solubility if the pure acetone is used. The solvent in the granulation process allows, that (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) can be introduced in the process for preparing an amorphous solid dispersion (ASD) in form of the amorphous form or the crystalline modification I or in a mixture of both forms.

c) The Manufacturing Process

It was surprisingly found that an amorphous solid dispersion (ASD) according to the present invention comprising active ingredient (I) could not be manufactured by hot molt extrusion as proposed and favored by C. Leuner and J. Dressman. Applying a non-solvent method such as hot melt extrusion resulted in a racemic mixture of active ingredient (I). As only one enantiomer of active ingredient (I) is effective in-vivo, a decrease in bioavailability would be observed after administration of an amorphous solid dispersion comprising active ingredient (I) manufactured by hot melt extrusion to an animal or a human. In contrast to the prior art an amorphous solid dispersion comprising active ingredient (I) according to the present invention could be manufactured by a wet granulation method, which safeguarded the enantiomeric purity in a reproducible way in every batch manufactured.

Amorphous Solid Dispersion (ASD)

The present invention provides an amorphous solid dispersion (ASD) comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix.

The present invention also provides an amorphous solid dispersion (ASD) comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix and optionally sweeteners, flavoring agents and colorants.

The present invention also provides an amorphous solid dispersion (ASD) wherein (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) is present in amorphous form.

In the context of the present invention the pharmaceutically acceptable matrix consists of the combination of the solid dispersion base and the carrier.

In the context of the present invention the solid dispersion base is a pharmaceutically acceptable polymer, selected from the group consisting of polyethylene oxide, polyvinylpyrrolidone (PVP), vinylpyrrolidone/vinylacetate copolymer (copovidone) (e.g. Kollidon VA64), polyalkylene glycol (e.g. polyethylene glycol), hydroxyalkyl cellulose (e.g. hydroxypropyl cellulose), hydroxyalkyl methyl cellulose (e.g. hydroxypropyl methyl cellulose), carboxymethyl cellulose, sodium carboxymethyl cellulose, polymethacrylates (e.g. Eudragit® types), polyvinyl alcohol, polyvinyl acetate, vinyl alcohol/vinyl acetate copolymer, and combinations thereof. Preferred the solid dispersion base is selected from the group consisting of polyvinylpyrrolidone (PVP), vinylpyrrolidone/vinylacetate copolymer (copovidone), hydroxypropyl cellulose, hydroxypropyl methyl cellulose, polyethylene glycol and polyethylene oxide. Very preferred the solid dispersion base is the polymer polyvinylpyrrolidone (PVP).

In the context of the present invention the carrier is selected from the groups of fillers, lubricants, disintegration promoters, surfactants, sweeteners, flavoring agents and/or colorants or a combination thereof.

(4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) and the solid dispersion base are present in the amorphous solid dispersion (ASD) in a ratio of active ingredient (I) to solid dispersion base of 1 to 0.5 up to 1 to 20. Preferred is a ratio of active ingredient (1) to solid dispersion base of 1 to 0.5 up to 1 to 10, more preferred is a ratio of active ingredient (I) to solid dispersion base of 1 to 0.5 up to 1 to 5 and very preferred is a ratio of active ingredient (I) to solid dispersion base of 1 to 2. Especially a ratio of active ingredient (I) to solid dispersion base of 1 to 2 enables high drug-load and small tablet sizes.

The drug-load of active ingredient (I) in the amorphous solid dispersion (ASD) is >20% and preferred the drug-load in the amorphous solid dispersion (ASD) is ≥25%.

(4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) and the carrier is present in the amorphous solid dispersion (ASD) in a ratio of active ingredient (I) to carrier of 1 to 0 up to 1 to 20. Preferred is a ratio of active ingredient (I) to carrier of 1 to 0 to 1 to 5 and very preferred is a ratio of active ingredient (I) to carrier of 1 to 1. This means that also no carrier can be available in the matrix.

Solid Pharmaceutical Dosage Forms

The present invention provides solid pharmaceutical dosage forms for oral administration comprising an amorphous solid dispersion (ASD), containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix.

The present invention provides solid pharmaceutical dosage forms for oral administration comprising an amorphous solid dispersion (ASD), containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix and optionally sweeteners, flavoring agents and colorants.

The present invention provides solid pharmaceutical dosage forms for oral administration comprising an amorphous solid dispersion (ASD), containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix, and further pharmaceutical acceptable excipients.

The present invention provides solid pharmaceutical dosage forms for oral administration comprising an amorphous solid dispersion (ASD), containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix and optionally sweeteners, flavoring agents and colorants, and further pharmaceutical acceptable excipients.

The present invention also provides solid pharmaceutical dosage forms for oral administration comprising
a) an amorphous solid dispersion (ASD), containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix,
b) at least one lubricant,
c) at least one disintegration promoter,
d) optionally one or more fillers, and
e) optionally one or more surfactants.

Furthermore, the present invention provides solid pharmaceutical dosage forms for oral administration comprising
a) an amorphous solid dispersion (ASD), containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix,
b) at least one lubricant,
c) at least one disintegration promoter,
d) optionally one or more fillers, and
e) optionally one or more surfactants,

wherein at least 85% of active ingredient (I) are released into the release medium after 30 minutes, according to the release method of the European Pharmacopoeia using apparatus 2 (paddle).

For oral administration, the amorphous solid dispersion comprising the active ingredient (I) can be formulated into solid or liquid preparations such as powder, granulates, pellets, tablets, sachets, capsules, dragees, chewable tablets, effervescent tablets, dispersible tablets, troches, lozenges, melts, solutions, suspensions, or emulsions, and may be prepared according to the methods known to the art of the manufacture of pharmaceutical compositions.

The pharmaceutical dosage form according to the present invention is a tablet.

The pharmaceutical dosage form according to the present invention is an immediate release tablet.

The pharmaceutical dosage form according to the present invention is a tablet optionally covered with a coating, preferably the tablet is covered with a coating.

The drug-load of active ingredient (I) in the tablet is >20% and preferred the drug-load in the tablet is ≥23%.

The pharmaceutical dosage form according to the present invention is also an amorphous solid dispersion (ASD), containing active ingredient (I) in a matrix and optionally sweeteners, flavoring agents and colorants, formulated into sachets.

(4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) is present in a tablet in an amount of 2 mg up to 100 mg, preferred in an amount of 5 mg up to 50 mg, also preferred in an amount of 20 mg up to 50 mg, more preferred in an amount of 50 mg.

The solid pharmaceutical dosage form, especially in form of a tablet, as well as the granulates of the amorphous solid dispersion (ASD) are expected to be storage stable for an extended period of time, preferably long-term stable.

The solid pharmaceutical dosage form, especially in form of a tablet, as well as the granulates of the amorphous solid dispersion (ASD) are storage stable for at least 3 months, preferred for at least 6 months, also preferred for at least 12 months, also preferred for at least 24 months, also preferred for at least 30 months and more preferred for at least 48 months.

Long-term storage means storage for more than 24 months.

Storage stable means stable with a maximum of 10% degradation of the active ingredient (I), preferred with a maximum of 3% degradation of the active ingredient (I), and with preservation of the amorphous form of the active ingredient (I).

Storage conditions for evaluation of the stability are in example closed container 25° C./60% relative humidity or closed container 30° C./75% relative humidity or open container 25° C./60% relative humidity or open container 40° C./75% relative humidity (stress conditions).

The surprisingly excellent stability behavior of the solid pharmaceutical dosage form containing active ingredient (I), even at stress conditions (open storage at 40° C. and 75% relative humidity for 6 months) allows for non-protective packaging (e.g HPDE bottles without desiccant) of the pharmaceutical dosage form of active ingredient (I).

In the context of the present invention, immediate release tablets are particularly those which have released at least 85% of active ingredient (I) into the release medium after 30 minutes, according to the release method of the European Pharmacopoeia using apparatus 2 (paddle). The rotation speed of the stirrer is 75 rpm (revolutions per minute) in 900 ml release medium.

According to the present invention the release medium is acetate buffer pH 4.5+0.1% SDS or +0.15% SDS or +0.2% SDS or +0.3% SDS, or of 0.01 M hydrochloric acid+0.1% SDS or +0.2% SDS. SDS is the abbreviation for sodium dodecyl sulfate also called sodium lauryl sulfate.

The present invention further relates to the use of (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (I) for preparing a solid pharmaceutical dosage form for oral administration according to the invention.

The active ingredient (I) is present in the pharmaceutical dosage forms according to the invention in amorphous form.

The present invention provides solid pharmaceutical dosage forms, wherein the amorphous solid dispersion is substantially homogeneous.

In the context of the present invention, “excipients” are fillers, lubricants, disintegration promoters, surfactants, sweeteners, flavoring agents and colorants. It may therefore come to happen that a person skilled in the art assigns similar or even identical substances to be member of more than one of the above-mentioned groups of substances. Within the context of the present invention, the functional descriptions of the substances are however intentionally filled with specific substances to clarify their respective property assigned to them.

The expression ‘pharmaceutically acceptable’ refers to those excipients, within the scope of sound medical judgment, suitable for use in contact with the tissues of human beings and animals without excessive toxicity, irritation, allergic response, or other problems or complications, commensurate with a reasonable benefit/risk ratio.

Fillers that can be used in the formulation according to the present invention are those selected from the list consisting of cellulose powder, microcrystalline cellulose, silicified microcrystalline cellulose, dicalcium phosphate, tricalcium phosphate, magnesium trisilicate, mannitol, maltitol, sorbitol, xylitol, lactose (anhydrous or as a hydrate, for example monohydrate), dextrose, maltose, sucrose, glucose, fructose or maltodextrins. Preferred as filler is microcrystalline cellulose or lactose or a combination thereof. Very preferred is that no filler is used.

Lubricants prevent ingredients from sticking, e.g. to production equipment. Lubricants that can be used in the formulation according to the present invention are those selected from the list consisting of magnesium stearate, sodium stearylfumarate, stearic acid, glycerin monostearate, glycerin monobehenate, calcium behenate, hydogenated vegetable fat or oil, polyethylenglycol and talc. Preferred lubricants according to the present invention are those selected from the list consisting of magnesium stearate, stearic acid and talc. Very preferred as lubricant is magnesium stearate.

Disintegration promoter expand and dissolve when wet. They can be used to break the dosage form apart in the digestive tract, releasing the active ingredients. Disintegration promoters suitable in the context of the present invention are those selected from the list consisting of alginic acid, cross-linked polyvinylpyrrolidone, maize starch, modified starch, and starch derivatives such as sodium carboxymethyl starch, cellulose derivatives such as carmellose calcium (carboxymethylcellulose calcium) and croscarmellose sodium (cross-linked polymer of carboxymethylcellulose sodium) or microcrystalline cellulose or a combination of croscarmellose sodium and microcrystalline cellulose. Preferred as a disintegration promoter is croscarmellose sodium or cross-linked polyvinylpyrrolidone. Very preferred as a disintegration promoter is croscarmellose sodium.

Surfactants are usually organic compounds that are amphiphilic, meaning they contain both hydrophobic groups (their tails) and hydrophilic groups (their heads). Therefore, a surfactant contains both a water-insoluble (or oil-soluble) component and a water-soluble component and help to solubilize certain chemical compounds. Surfactants according to the present invention are complexing agents such as cyclodextrines and sodium ethylene diamintetraacetic acid (EDTA), cosolvents such as ethanol, propylene glycol and dimethyl acetamide, tensides such as fatty alcohols (e.g. cetylalcohol), phospholipids (e.g. lecithine), bile acids, polyoxyethylene stearate fat esters (e.g. polyoxyethylene), polyoxyethylene sorbitan fat esters, polyoxypropylene-polyoxyethylene-block copolymers (e.g. Poloxamer), alkylsulfates (e.g. sodium lauryl sulfate, sodium cetylstearyl sulfate), alkyl soaps (e.g. sodium palmitate, sodium stearate) and saccharose fatty acid esters. Preferred as surfactant is sodium lauryl sulfate.

Preferred as sweetener is a pharmaceutically acceptable excipients that has a similar taste to sugar. Sweeteners suitable in the context of the present invention are those selected from the list consisting of sucralose, saccharin, sodium-, potassium- or calcium saccharin, potassium acesulfame, neotame, alitame, glycyrrhizin or thaumatin, or sugars such as glucose, mannitol, fructose, saccharose, maltose, maltitol, galactose, sorbitol or xylitol. In the context of the present invention sweeteners are added in amounts known for persons skilled in the art.

In the context of the present invention flavoring agents are pharmaceutically acceptable excipients appropriate to improve or give an agreeable taste of a pharmaceutical dosage form to complement its effect and also to increase its elegance. In the context of the present invention flavoring agents are natural flavoring substances obtained from plant or animal raw materials, nature-identical flavoring substances obtained by synthesis or isolated through chemical processes, which are chemically and organoleptically identical to flavoring substances naturally present in products intended for human consumption and artificial flavoring substances. In the context of the present invention flavoring agents are added in amounts known for persons skilled in the art. Flavoring agents suitable in the context of the present invention are those selected from the list consisting of synthetic/artificial flavoring agents such as amyl acetate (banana flavoring), benzaldehyde (cherry or almond flavor), ethyl butyrate (pineapple), methyl anthranilate (grape), natural flavoring agents such as essential oils and oleoresins, herbs and spices, and natural-identical flavoring agents which are flavoring substances that are obtained by synthesis or are isolated through chemical processes and whose chemical make-up is identical to their natural counterpart. In the context of the present invention flavoring agents are added in amounts known for persons skilled in the art.

In the context of the present invention colorants are pharmaceutically acceptable excipients appropriate to color an uncolored pharmaceutical dosage form or to enhance its color, to minimize batch-to-batch variations or to replace a color already present to complement its effect and also to increase its elegance. It can be any dyes, lakes or pigment such as indigo carmine, riboflavine and titanium dioxide. In the context of the present invention colorants are added in amounts known for persons skilled in the art.

In the context of the present invention the optional coating is carried out with addition of customary coating and film-forming agents familiar to the person skilled in the art, such as hydroxy-propylcellulose, hydroxypropylmethylcellulose (Hypromellose), ethylcellulose, polyvinylpyrrolidone, vinylpyrrolidone-vinyl acetate copolymers (for example Kollidon® VA64, BASF), shellac, acrylic and/or methacrylic acid ester copolymers with trimethylammonium methylacrylate, copolymers of dimethylaminomethacrylic acid and neutral methacrylic acid esters, polymers of methacrylic acid or methacrylic acid esters, ethyl acrylate-methyl methacrylate copolymers, methacrylic acid-methyl acrylate copolymers, propylene glycol, polyethylene glycol (e.g. polyethylene glycol 3350), glycerol triacetate or triethyl citrate, and/or colorants/pigments such as, for example, titanium dioxide, iron oxide (e.g. red iron oxide, yellow iron oxide), indigotin or suitable colour lakes, and/or antitacking agents such as talc, and/or opacifiers such as titanium dioxide. As optional coating and film-forming agents according to the present invention, Hypromellose and polyethylene glycol are preferred, as colorant iron oxide red is preferred and as opacifier titanium dioxide is preferred.

A mixture of the coating substances mentioned herein may also be used as a ready-to-use coating system such as commercially available under the trade name Opadry®. Opadry 14F94373@ is a mixture of about 60 wt. % hydroxypropylmethylcellulose, about 19.4 wt. % titanium dioxide, about 0.6 wt. % ferric oxide red and about 20 wt. % polyethylene glycol. The ready-to-use coating system available under the trade name Opadry® is preferred.

Preferably the coating is about 0.5% to 10% by weight of the coated tablet formulation, preferably 0.5% to 4.5% by weight of the coated tablet formulation, more preferably about 1.5% to 4.5% by weight of the coated tablet formulation.

Binders are used in the comparison formulations according to the invention. Binders that can be used are cellulose powder, microcrystalline cellulose, silicified microcrystalline cellulose, dicalcium phosphate, tricalcium phosphate, magnesium trisilicate, mannitol, maltitol, sorbitol, xylitol, lactose (anhydrous or as a hydrate, for example monohydrate), dextrose, maltose, sucrose, glucose, fructose, maltodextrins or hypromellose (e.g. hypromellose 3 cP). Preferred as binder is hypromellose (e.g. hypromellose 3 cP).

Manufacturing Process of the Amorphous Solid Dispersion (ASD)

The present invention provides a process for preparing an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that the amorphous solid dispersion (ASD) is prepared by wet granulation.

The present invention provides a process for preparing an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that the amorphous solid dispersion (ASD) is prepared by fluidized bed granulation.

The wet granulation can be carried out in a mixer, in a spray dryer or in a fluidized bed granulator. The wet granulation in a fluidized bed granulator (=fluidized bed granulation) is preferred.

In the wet granulation the active ingredient (I) is solved in the granulating fluid and introduced into the fluidized bed granulator. Most preferably, the granulating fluid containing the active ingredient (I) is sprayed onto a carrier via fluidized bed granulation.

In the context of the present invention the granulating fluid consists of the solid dispersion base and solvents.

Solvents suitable for manufacturing the amorphous solid dispersions by solvent evaporation processes such as fluidized bed granulation can be any solvent, wherein the active ingredient (I) can be dissolved. The polymer of the solid dispersion base has also to be sufficiently soluble to make the process practicable. Preferred solvents include alcohols (e.g. methanol, ethanol, n-propanol, isopropanol, and butanol), ketones (e.g. acetone, methyl ethyl ketone and methyl isobutyl ketone), esters (e.g. ethyl acetate and propyl acetate) and various other solvents such as acetonitrile, methylene chloride, chloroform, hexane, toluene, tetrahydrofuran, cyclic ethers, and 1,1,1-trichloroethane. Lower volatility solvents, such as dimethyl acetamide or dimethyl sulfoxide can also be used. Preferred solvents for manufacturing the amorphous solid dispersions comprising the active ingredient (I) are methanol, ethanol, n-propanol, isopropanol, acetone or mixtures thereof. Also preferred for manufacturing the amorphous solid dispersions comprising the active ingredient (I) is ethanol or a mixture of 20% ethanol and 80% acetone or 50% ethanol and 50% acetone. Very preferred for manufacturing the amorphous solid dispersions comprising the active ingredient (I) is a mixture of 50% ethanol and 50% acetone.

The present invention provides a process for preparing an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that the amorphous solid dispersion (ASD) is prepared by fluidized bed granulation, in which the active ingredient (I) is solved in the granulating fluid and introduced into the fluidized bed granulator.

The present invention provides a process for preparing an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that the amorphous solid dispersion (ASD) is prepared by fluidized bed granulation, in which the active ingredient (I) is solved in the granulating fluid, which contains a mixture of 50% ethanol and 50% acetone, and is introduced into the fluidized bed granulator.

The amorphous solid dispersion (ASD) is preferably isolated as granulate.

The present invention provides a process for preparing an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that
a) (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) is dissolved in a suitable solvent or solvent mixture,
b) the polymer (solid dispersion base) is added to receive the granulating liquid,
c) the granulating liquid is sprayed onto the carrier,
d) the solvent or the solvents are evaporated to result in a granulate.

In an additional step e) the from step d) resulting granulate is optionally further processed by mixing with sweeteners, flavoring agents and colorants and/or milling and/or sieving and/or compacting to result in a granulate which can be used as solid pharmaceutical dosage form.

Amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) producible by one of the methods mentioned above.

Amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) produced by one of the methods mentioned above.

Manufacturing Process of the Solid Pharmaceutical Dosage Forms

The present invention provides a process for preparing solid pharmaceutical dosage forms for oral administration comprising an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that
a) an amorphous solid dispersion (ASD), comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix, is initially prepared,
b) and the amorphous solid dispersion (ASD), optionally with addition of pharmaceutically acceptable excipients, is then converted into the pharmaceutical dosage form.

Furthermore the present invention provides a process for preparing solid pharmaceutical dosage forms for oral administration comprising an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that
a) an amorphous solid dispersion (ASD), comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix, is initially prepared,
b) and the amorphous solid dispersion (ASD), optionally with addition of pharmaceutically acceptable excipients, is then converted into the pharmaceutical dosage form,

wherein at least 85% of active ingredient (I) are released into the release medium after 30 minutes, according to the release method of the European Pharmacopoeia using apparatus 2 (paddle).

Furthermore the present invention provides a process for preparing solid pharmaceutical dosage forms for oral administration comprising an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that
a) an amorphous solid dispersion (ASD), comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix, is prepared by wet granulation,
b) and the amorphous solid dispersion (ASD), optionally with addition of pharmaceutically acceptable excipients, is then converted into the pharmaceutical dosage form.

The solvent in the granulation process of preparing an amorphous solid dispersion (ASD) allows, that (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) can be introduced in the process for preparing solid pharmaceutical dosage forms in form of the amorphous form or the crystalline modification I or in a mixture of both forms.

Converted into the pharmaceutical dosage form in process step (b) comprises, for example, tabletting, filling into capsules, preferably hard gelatine capsules, or filling as sachets, in each case according to customary methods familiar to the person skilled in the art, if appropriate with addition of further pharmaceutically suitable excipients.

For conversion into the solid pharmaceutical dosage form in process step (b) the amorphous solid dispersion, which is isolated as a granulate, can be roller compacted and grinded with and without further excipients to obtain roller compacted granulate. The obtained granulate with and without further excipients is compressed into the pharmaceutical dosage form such as tablets.

Furthermore the present invention provides a process for preparing solid pharmaceutical dosage forms for oral administration comprising an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that
a) an amorphous solid dispersion (ASD), comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix, is prepared by wet granulation,
b) to the amorphous solid dispersion (ASD) further pharmaceutically acceptable excipients are added,
c) the resulting mixture is compressed into tablets, and
d) the tablets are optionally coated to receive the pharmaceutical dosage form.

Furthermore the present invention provides a process for preparing solid pharmaceutical dosage forms for oral administration comprising an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that
a) an amorphous solid dispersion (ASD), comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix, is prepared by wet granulation,
b) to the amorphous solid dispersion (ASD) further pharmaceutically acceptable excipients are added,
c) the resulting mixture is roller compacted and optionally grinded,
d) optionally further pharmaceutically acceptable excipients are added,
e) the resulting mixture is compressed into tablets, and
f) the tablets are optionally coated to receive the pharmaceutical dosage form.

Solid pharmaceutical dosage forms for oral administration comprising an amorphous solid dispersion (ASD) containing (4S)-2⁴-chloro-4-ethyl-7³-fluoro-3⁵-methoxy-3²,5-dioxo-14-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) producible by one of the methods mentioned above.

Solid pharmaceutical dosage forms for oral administration comprising an amorphous solid dispersion (ASD) containing (4S)-2⁴-chloro-4-ethyl-7³-fluoro-3⁵-methoxy-3²,5-dioxo-14-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) produced by one of the methods mentioned above.

Medicaments and Use

Administration of the oral solid dosage form comprising an amorphous solid dispersion of amorphous active ingredient (I) stabilized by selected excipients and manufactured by a process which ensures enantiomeric purity leads to high relative bioavailability in human ranging from 85% up to even 100%, preferred ranking from 88% up to even 100%.

The present invention further provides medicaments comprising a solid pharmaceutical dosage form for oral administration in accordance with the invention comprising the active ingredient (I).

The present invention further relates to the use of solid pharmaceutical dosage forms for oral administration in accordance with the invention comprising the active ingredient (I) and for preparing a medicament for the treatment and/or prophylaxis of disorders, preferably thrombotic or thromboembolic disorders and/or thrombotic or thromboembolic complications, in particular cardiovascular disorders including coronary artery disease, angina pectoris, myocardial infarction or stent thrombosis, as well as disorders in the cerebrovascular arteries and other disorders, leading to transitory ischaemic attacks (TIA), ischemic strokes including cardioembolic as well as non-cardioembolic strokes, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

The present invention further relates to the use of solid pharmaceutical dosage forms for oral administration in accordance with the invention comprising the active ingredient (I) for prophylaxis, secondary prophylaxis and/or treatment of disorders, particularly myocardial infarction, ischemic strokes including cardioembolic as well as non-cardioembolic strokes, acute limb ischemia, reocclusions and restenoses after interventions such as angioplasty, stent implantation or surgery and bypass, and/or stent thrombosis.

Below, the invention is illustrated in detail by preferred working examples; however, the invention is not limited to these examples. Unless indicated otherwise, all amounts given refer to mg of dosage form.

### EXPERIMENTAL PART

### Abbreviations

PXRD: powder X-ray diffraction
IR tablet: immediate release tablet
rel. BA: relative bioavailability
AUC/D: Area under the curve per dose
Cmax/D: maximum concentration per dose
HPMCAS MG: AquaSolve™ Hydroxypropylmethylcellulose Acetate Succinate Typ MG
SDS: sodium dodecyl sulfate also called sodium lauryl sulfate
rh: relative humidity
initial: earliest timepoint of analysis after manufacturing
Active ingredient (I) crystalline means active ingredient (I) in crystalline modification I.
If “open” or “closed” is not mentioned in regard to storing conditions, then closed storage is meant.

### 1. Preparation of Active Ingredient (I)

### 1.1 Preparation of Active Ingredient (I) in Amorphous Form

Active ingredient (I) is (4S)-2⁴-chloro-4-ethyl-7³-fluoro-3⁵-methoxy-3²,5-dioxo-14-(trifluoromethyl)-3²H-6-aza-3(4,1)-pyridina-1(1)-[1,2,3]triazola-2(1,2),7(1)-dibenzenaheptaphane-7⁴-carboxamide, also named as 4-({(2S)-2-[4-{5-chloro-2-[4-(trifluoromethyl)-1H-1,2,3-triazol-1-yl]phenyl}-5-methoxy-2-oxopyridin-1(2H)-yl]butanoyl}amino)-2-fluorobenzamide, which can be prepared according to WO 2017/005725 Example 234 and Example 235 in the amorphous form.

### 1.2 Preparation of Active Ingredient (I) in Crystalline Modification I

Active ingredient (I) in crystalline modification I can be obtained by dissolving the active ingredient (I) in the amorphous form in an inert solvent and crystallising the active ingredient (I) in the crystalline modification I with a seed of the compound of the formula (II) in the crystalline modification A.

Solubility of the active ingredient (I) in crystalline modification I in ethanol is approximately 35 mg/ml, in acetone it is approximately 80 mg/ml and in a mixture of ethanol/acetone (1:1) it is approximately 180 mg/ml.

### 1.2.1 Preparation of 4-({(2S)-2-[4-{3-Chloro-2-fluoro-6-[4-(trifluoromethyl)-1H-1,2,3-triazol-1-yl]phenyl}-5-methoxy-2-oxopyridin-1(2H)-yl]propanoyl}amino)-2-fluoro-benzamide (compound of the formula (II))

### 1.2.1.1 1-(2-Bromo-4-chloro-3-fluorophenyl)-4-(trifluoromethyl)-1H-1,2,3-triazole

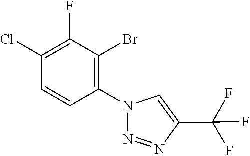 [SMILES: Fc1c(Cl)ccc(-n2cc(C(F)(F)F)nn2)c1Br]

1-(2-Bromo-4-chloro-3-fluorophenyl)-4-(trifluoromethyl)-1H-1,2,3-triazole is synthesized starting with 2-bromo-4-chloro-3-fluoroaniline (WO 2016/168098, page 59-60) by first generating the azido derivative (in the presence of tert-butyl nitrite and trimethylsilyl azide, in analogy to the synthesis of example 2.18A, WO 2017/005725, page 92-93) and second performing a cycloaddition of the azido derivative with trifluoropropyne (in the presence of copper(I) oxide, in analogy to the synthesis of example 2.26A, WO 2017/005725, page 102).

### 1.2.1.2 4-{3-Chloro-2-fluoro-6-[4-(trifluoromethyl)-1H-1,2,3-triazol-1-yl]phenyl}-2,5-dimethoxypyridine

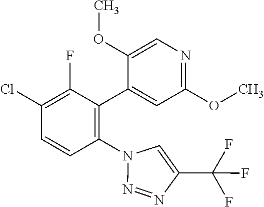 [SMILES: COc1cc(-c2c(-n3cc(C(F)(F)F)nn3)ccc(Cl)c2F)c(OC)cn1]

A mixture of 1-(2-bromo-4-chloro-3-fluorophenyl)-4-(trifluoromethyl)-1H-1,2,3-triazole (982 mg, 2.85 mmol), (2,5-dimethoxypyridin-4-yl)boronic acid (WO 2019/175043, page 23-24) (626 mg, 3.42 mmol, 1.2 eq.) and potassium carbonate (1.18 g, 8.55 mmol, 3.0 eq.) was dissolved in 1,4-dioxane (50 ml) and flushed with argon for 10 min before [1,1-bis(diphenylphosphino)ferrocene]palladium(II) chloride monodichloromethane adduct (233 mg, 0.29 mmol, 0.1 eq.) was added. The reaction mixture was stirred at 100° C. (oil bath already pre-heated to 100° C.) overnight. Additional (2,5-dimethoxypyridin-4-yl)boronic acid (209 mg, 1.14 mmol, 0.4 eq.) and [1,1-bis(diphenylphosphino)ferrocene]palladium(II) chloride monodichloromethane adduct (116 mg, 0.14 mmol, 0.05 eq.) were added. The reaction mixture was stirred at 100° C. for additional 5 h, left at RT for the weekend and filtered through Celite© which was washed with 1,4-dioxane. The combined filtrates were concentrated under reduced pressure.

The residue was purified by chromatography (silica gel, eluent: cyclohexane/ethyl acetate gradient). Yield: 432 mg (38% of theory).

LC-MS (method 2): Rₜ=2.13 min; MS (ESIpos): m/z=403 [M+H]⁺

¹H-NMR (400 MHz, DMSO-d₆): δ [ppm]=9.17/9.16 (2×s, 1H), 8.03/8.01 (2×d, 1H), 7.86 (s, 1H), 7.75/7.75 (2×d, 1H), 6.82 (s, 1H), 3.79 (s, 3H), 3.54 (s, 3H).

### 1.2.1.3 4-{3-Chloro-2-fluoro-6-[4-(trifluoromethyl)-1H-1,2,3-triazol-1-yl]phenyl}-5-methoxy-pyridin-2(1H)-one

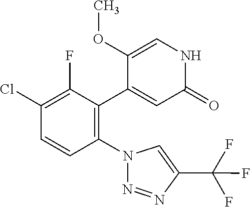 [SMILES: COc1c[nH]c(=O)cc1-c1c(-n2cc(C(F)(F)F)nn2)ccc(Cl)c1F]

Pyridine hydrobromide (429 mg, 2.68 mmol, 2.5 eq.) was added to a solution of 4-{3-chloro-2-fluoro-6-[4-(trifluoromethyl)-1H-1,2,3-triazol-1-yl]phenyl}-2,5-dimethoxypyridine (432 mg, 1.07 mmol) in N,N-dimethylformamide (10 ml). The mixture was stirred at 100° C. overnight and concentrated under reduced pressure. The residue was dissolved in water. After addition of ethyl acetate and phase separation, the aqueous phase was extracted two times with ethyl acetate. The combined organic phases were dried over anhydrous sodium sulfate, filtered and concentrated under reduced pressure. The residue was purified by chromatography (silica gel, eluent: dichloromethane/methanol gradient). Yield: 285 mg (68% of theory).

LC-MS (method 2): Rₜ=1.46 min; MS (ESIpos): m/z=389 [M+H]⁺

¹H-NMR (600 MHz, DMSO-d₆): δ [ppm]=11.3 (br s, 1H), 9.23 (s, 1H), 8.10-7.99 (m, 1H), 7.77 (m, 1H), 7.15 (s, 1H), 6.41 (s, 1H), 3.45 (s, 3H).

### 1.2.1.4 4-({(2S)-2-[4-{3-Chloro-2-fluoro-6-[4-(trifluoromethyl)-1H-1,2,3-triazol-1-yl]phenyl}-5-methoxy-2-oxopyridin-1(2H)-yl]propanoyl}amino)-2-fluorobenzamide (compound of the formula (II)

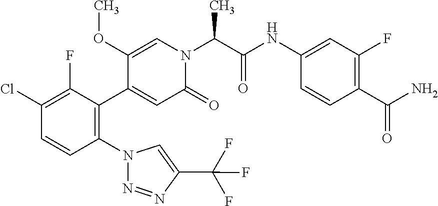 [SMILES: COc1cn([C@@H](C)C(=O)Nc2ccc(C(N)=O)c(F)c2)c(=O)cc1-c1c(-n2cc(C(F)(F)F)nn2)ccc(Cl)c1F]

1,1,3,3-Tetramethylguanidine (420 μl, 3.35 mmol, 3.0 eq.) was added under argon atmosphere at RT to a solution of 4-{3-chloro-2-fluoro-6-[4-(trifluoromethyl)-1H-1,2,3-triazol-1-yl]phenyl}-5-methoxypyridin-2(1H)-one (438 mg, 1.12 mmol) in 2-propanol/acetone (4:1, 7.5 ml). The mixture was stirred at RT for 15 min, followed by addition of 4-{[(2R)-2-bromopropanoyl]amino}-2-fluorobenzamide (WO 2020/127504, example 1.19A, page 76) (355 mg, 1.23 mmol, 1.1 eq.) and further 2-propanol/acetone (4:1, 7.5 ml). The reaction mixture was stirred at RT overnight and concentrated under reduced pressure. The residue was purified by chromatography (silica gel, eluent: dichloromethane/methanol gradient) and preparative HPLC (reversed phase, eluent: acetonitrile/water gradient). Yield: 539 mg (81% of theory).

LC-MS (method 2): Rₜ=1.65 min; MS (ESIpos): m/z=597 [M+H]⁺

¹H-NMR (500 MHz, DMSO-d₆): δ [ppm]=10.72/10.63 (2×s, 1H), 9.24/9.13 (2×s, 1H), 8.06-7.99 (m, 1H), 7.79-7.74 (m, 1H), 7.72-7.60 (m, 2H), 7.56-7.48 (m, 2H), 7.38-7.32 (m, 1H), 7.27/7.25 (2×s, 1H), 6.48/6.47 (2×s, 1H), 5.51-5.44 (m, 1H), 3.47/3.45 (2×s, 3H), 1.65/1.64 (2×s, 3H).

### 1.2.2 Preparation of the Compound of the Formula (II) in Crystalline Modification A

306 mg of compound of the formula (II) in amorphous form was dissolved in 20 mL of a mixture of 50 vol.-% ethanol and 50 vol.-% water at room temperature. The solution was stirred 24 hours at room temperature, resulting in the precipitation of a white solid. The solvent was evaporated in a rotary evaporator. The obtained solid was dried in a vacuum oven at 40° C. for 16 hours. 273 mg of compound of the formula (II) in the crystalline modification A was obtained.

### 1.2.3 Preparation of the Active Ingredient (I) in Crystalline Modification I

30 mg of the active ingredient (I) in amorphous form was dissolved in 2 mL of ethanol at room temperature. 660 μL of water was added to the solution dropwise until a cloudy solution was observed. The solution was then seeded with 1 mg of crystalline modification A of compound of the formula (II). Shortly after seeding, the precipitation of further small particles was observed, but the particles rapidly disappeared upon stirring, resulting in a seemingly clear solution. After stirring at room temperature for 48 hours, a suspension was obtained. The solid was filtered under vacuum and dried overnight under ambient conditions. The XRPD pattern of the obtained solid corresponds to the crystalline modification I of the active ingredient (I). The ¹H-NMR analysis of the resulting solid indicates that the solid contained approximately 5 wt-% of compound of the formula (II).

### 1.2.4 Preparation of the Active Ingredient (I) in Crystalline Modification I as Pure Active Ingredient

20.0 g of the active ingredient (I) in amorphous form was dissolved in a mixture of 40.0 g of propan-2-ol and 10.0 g of acetone, at room temperature. The mixture was heated up to 60° C. and to the resulting solution 126.0 g of water was added during 60 minutes. The resulting mixture was seeded with 100.0 mg of crystalline modification I of the active ingredient (I) and stirred at 60° C. for 3 hours. Additional 4.8 g of the active ingredient (I) in amorphous form was then added and the mixture was stirred at 60° C. overnight. The resulting suspension was cooled down to 20° C. in 60 minutes and stirred at 20° C. for 90 minutes. So-obtained suspension was filtered under vacuum, washed twice with 42.5 g of propan-2-ol:acetone:water mixture in the mass ratio 4:1:12 and dried in vacuum, at 40° C. Yield: 22.4 g (90.3% of theoretical yield) of pale-white solid.

### 2. Release/Dissolution Method

According to the European Pharmacopoeia, 10th Edition, last revision of monograph 01/2016, the oral solid dosage form is tested with apparatus 2 (paddle). The rotation speed of the stirrer is 75 rpm (revolutions per minute) in 900 ml of the medium listed below. The release criterion is then fulfilled if all 6 test specimens have released at least 85% of active ingredient (I) into the release medium after an investigation period of 30 minutes.

| Medium | Used for Examples |
|---|---|
| Acetate buffer pH 4.5 + 0.1% SDS | 3-1 to 3-9 and 4-1 to 4-3 |
| Acetate buffer pH 4.5 + 0.2% SDS | 4-7, 4-8, 4-10, 4-11 and 4-13 |
| Acetate buffer pH 4.5 + 0.3% SDS | 4-4 to 4-6, 4-9, 4-12 and 4-14 |
| | to 4-18 |
| Acetate buffer pH 4.5 + 0.15% SDS | 4-14* |
| 0.01M hydrochloric acid + 0.1% SDS | 2-1 and 2-3 |
| 0.01M hydrochloric acid + 0.2% SDS | 2-2 |

*stability samples

### 3. Preparation Method for Liquid Formulations

### Example 1-1 (Comparison Example)

Mix tylose and water while stirring. Add the active ingredient (I) in crystalline modification I and keep stirring.

### Example 1-2 (Comparison Example)

Polyethylene glycol (PEG) solution was prepared by solving the active ingredient (I) in amorphous form in ethanol before the PEG is added. Water is added and the solution is mixed well.

### 4. Fluidized Bed Granulation Preparation Method

### Examples 2-1, 2-2 and 2-3 (Tablets as Comparison Examples)

The binder and the surfactant are dissolved in water and the active ingredient (I) is suspended in this solution. In the course of a fluidized bed granulation, this suspension is sprayed as granulating fluid on the initial charge composed of fillers and parts of the disintegration promoter. After drying and sieving the resulting granulates, the remaining parts of the disintegration promoter and a lubricant, which is optionally also magnesium stearate, are added and mixed. The ready to press blend thus obtained is compressed to produce tablets. The tablets are then coated with pigments which are suspended in an aqueous solution composed of coating and film-forming agents.

### Example 3-1 (Amorphous Solid Dispersion (ASD))

The solid dispersion base and active ingredient (I) are dissolved in organic solvent. In the course of a fluidized bed granulation, this solution is sprayed as granulating fluid on the initial charge composed of filler and the disintegration promoter (carrier). After drying and sieving the granulates are resulting. Organic solvents may be ethanol, acetone or combinations thereof.

### Examples 3-2, 3-3, 3-4, 3-5, 3-6, 3-7, 3-8 and 3-9 (Amorphous Solid Dispersion (ASD))

The solid dispersion base and active ingredient (I) are dissolved in organic solvent. In the course of a fluidized bed granulation, this solution is sprayed as granulating fluid on the initial charge of the disintegration promoter (carrier). After drying and sieving the granulates are resulting. Organic solvents may be ethanol, acetone or combinations thereof.

### Example 4-1 (Pharmaceutical Dosage Form (Tablet) Comprising Amorphous Solid Dispersion (ASD))

Granulates resulting from example 3-1 are blended together with added fillers and surfactant. This mixture is roller compacted and grinded followed by the addition and mixing of lubricant. The ready to press blend thus obtained is compressed to produce tablets. The tablets are then coated with pigments which are suspended in an aqueous solution composed of coating and film-forming agents. In the tablet the active ingredient (I) is present in an amount of 5 mg.

### Examples 4-2 and 4-3 (Pharmaceutical Dosage Forms (Tablets) Comprising Amorphous Solid Dispersion (ASD))

Granulates resulting from example 3-1 are blended together with added fillers, disintegration promoter and surfactant. This mixture is roller compacted and grinded followed by the addition and mixing of lubricant. The ready to press blend thus obtained is compressed to produce tablets. The tablets are then coated with pigments which are suspended in an aqueous solution composed of coating and film-forming agents. In the tablets the active ingredient (I) is present in an amount of 15 mg and 25 mg respectively.

### Examples 4-4, 4-5 and 4-6 (Pharmaceutical Dosage Forms (Tablets) Comprising Amorphous Solid Dispersion (ASD))

Granulates resulting from example 3-3 are blended together with filler, disintegration promoter and surfactant. Lubricant is added afterwards and mixed again. The ready to press blend thus obtained is compressed to produce tablets. In the tablets the active ingredient (I) is present in an amount of 50 mg.

### Examples 4-7, 4-8, 4-9 and 4-10 (Pharmaceutical Dosage Forms (Tablets) Comprising Amorphous Solid Dispersion (ASD))

Granulates resulting from example 3-4 or 3-5 are blended together with added disintegration promoter and surfactant. This mixture may be roller compacted and grinded. Afterwards lubricant is added to the mixture and mixed again. The ready to press blend thus obtained is compressed to produce tablets. The tablets may then be coated with pigments which are suspended in an aqueous solution composed of coating and film-forming agents. In the tablets the active ingredient (I) is present in an amount of 20 mg or 50 mg.

### Example 4-11 and 4-12 (Pharmaceutical Dosage Forms (Tablets) Comprising Amorphous Solid Dispersion (ASD))

Granulates resulting from example 3-5 may be roller compacted and grinded. Afterwards are blended together with lubricant. The ready to press blend thus obtained is compressed to produce tablets. The tablets may then be coated with pigments which are suspended in an aqueous solution composed of coating and film-forming agents. In the tablets the active ingredient (I) is present in an amount of 20 mg or 50 mg.

### Examples 4-13, 4-14, 4-15, 4-16, 4-17 and 4-18 (Pharmaceutical Dosage Forms (Tablets) Comprising Amorphous Solid Dispersion (ASD))

Granulates resulting from example 3-4, 3-5, 3-6, 3-7, 3-8 or 3-9 are blended together with added disintegration promoter. This mixture may be roller compacted and grinded. Afterwards lubricant is added to the mixture and mixed again. The ready to press blend thus obtained is compressed to produce tablets. The tablets may then be coated with pigments which are suspended in an aqueous solution composed of coating and film-forming agents. In the tablets the active ingredient (I) is present in an amount of 20 mg or 50 mg.

### 5. Hot Melt Extrusion Preparation Method

### Examples 5-1, 5-2, 5-4 and 5-5 (Grinded Extrudates as Comparison Examples)

Active ingredient (I), a surfactant and the solid dispersion base are mixed/blended. The mixture was extruded using a laboratory twin screw extruder at a temperature of 180° C. The extruded material may be cut and subsequently milled using an impact lab mill. The resulting granulate can be used as it is or it can be further formulated for example to sachet, capsule or tablet formulations.

### Examples 5-3 and 5-6 (Grinded Extrudates as Comparison Examples)

Active ingredient (I) and the solid dispersion base are mixed/blended. The mixture was extruded using a laboratory twin screw extruder at a temperature of 180° C. The extruded material may be cut and subsequently milled using an impact lab mill. The resulting granulate can be used as it is or it can be further formulated for example to sachet, capsule or tablet formulations.

### 6. Compositions of the Dosage Form in mg/Dosage Form

### 6.1 Liquid Formulation and Tablets as Comparison Examples (Table 1)

| | 1-1 | 1-2 | 2-1 | 2-2 | 2-3 |
|---|---|---|---|---|---|
| Active ingredient (I), crystalline | 0.6 | — | 5.0 | — | — |
| modification I | | | | | |
| Active ingredient (I), amorphous | — | 0.6 | — | 50.0 | 5.0 |
| form | | | | | |

Solid dispersion base (polymer)

| | | | | | |
|---|---|---|---|---|---|
| Polyvinylpyrrolidone (PVP) | — | — | — | — | — |
| HPMCAS MG | — | — | — | — | — |
| Kollidon VA64 | — | — | — | — | — |

Binder

| | | | | | |
|---|---|---|---|---|---|
| Hypromellose 3 cP | — | — | 3.40 | 15.4 | 3.40 |

Solvent

| | | | | | |
|---|---|---|---|---|---|
| Water | 954.4 | 499.7 | 68 | 440 | 68 |
| Ethanol 99% | — | 99.9 | — | — | — |
| Acetone | — | — | — | — | — |

Fillers

| | | | | | |
|---|---|---|---|---|---|
| Microcrystalline cellulose | — | — | 35.45 | 171.9 | 35.45 |
| Lactose monohydrate | — | — | 35.45 | 171.9 | 35.45 |
| Tylose | 5.0 | — | — | — | — |

Disintegration promoter

| | | | | | |
|---|---|---|---|---|---|
| Croscarmellose sodium | — | — | 4.25 | 22.0 | 4.25 |

Surfactant

| | | | | | |
|---|---|---|---|---|---|
| Sodium lauryl sulfate | — | — | 0.85 | 4.4 | 0.85 |
| PEG 4000 | — | — | — | — | — |
| PEG 6000 | — | — | — | — | — |
| PEG 400 | — | 399.8 | — | — | — |

Lubricant

| | | | | | |
|---|---|---|---|---|---|
| Magnesium stearate | — | — | 0.60 | 4.4 | 0.60 |

Coating and film-forming agents and colorants/pigments

| | | | | | |
|---|---|---|---|---|---|
| Hypromellose 5 cP | — | — | 1.50 | 5.0 | 1.50 |
| Hypromellose 15 cP | — | — | — | — | — |
| Polyethylene glycol 3350 | — | — | 0.30 | 1.0 | 0.30 |
| Red iron oxide | — | | 0.30 | 1.0 | 0.30 |
| Talc | — | — | 0.30 | 1.0 | 0.30 |
| Titanium dioxide | — | — | 0.60 | 2.0 | 0.60 |
| Total [mg] | 1000 | 1000 | 88 | 450 | 88 |
| Format (mm) | — | — | 6 | 16 × 7 | 6 |
| Disintegration time (min) | — | — | 3 | 6 | 5 |
| Dissolution at 30 min timepoint | — | — | 43 [42-44] | 88 [87-89] | 95 [94-95] |
| [%] | | | | | |
| F rel [%] animal | 11 | 100 | — | — | — |
| AUC/D animal [kg*h/L] | 0.491 | 4.43 | — | — | — |
| Cmax/D animal [kg/L] | 0.089 | 0.794 | — | — | — |
| PXRD (initial) | — | — | crystalline | amorphous | amorphous |

### 6.2 Amorphous Solid Dispersion (ASD) (Table 2)

| | 3-1 | 3-2 | 3-3 | 3-4 | 3-5 |
|---|---|---|---|---|---|
| Active ingredient (I), | — | — | 5.0 | — | — |
| crystalline modification I | | | | | |
| Active ingredient (I), | 5.0 | 5.0 | — | 5.0 | 5.0 |
| amorphous form | | | | | |

Solid dispersion base (polymer)

| | | | | | |
|---|---|---|---|---|---|
| Polyvinylpyrrolidone | 10.0 | 10.0 | 10.0 | 10.0 | 10.0 |
| (PVP) | | | | | |
| HPMCAS MG | — | — | — | — | — |
| Kollidon VA64 | — | — | — | — | — |

Binder

| | | | | | |
|---|---|---|---|---|---|
| Hypromellose 3 cP | — | — | — | — | — |

Solvent

| | | | | | |
|---|---|---|---|---|---|
| Water | — | — | — | — | — |
| Ethanol 99% | 60.0 | 30.0 | 6.0 | 6.0 | 15.0 |
| Acetone | — | — | 24.0 | 24.0 | 15.0 |

Fillers

| | | | | | |
|---|---|---|---|---|---|
| Microcrystalline | 7.5 | — | — | — | — |
| cellulose | | | | | |
| Lactose monohydrate | — | — | — | — | — |

Disintegration promoter

| | | | | | |
|---|---|---|---|---|---|
| Croscarmellose sodium | 7.5 | 5.0 | 5.0 | 5.0 | 5.0 |

Surfactant

| | | | | | |
|---|---|---|---|---|---|
| Sodium lauryl sulfate | — | — | — | — | — |
| PEG 4000 | — | — | — | — | — |
| PEG 6000 | — | — | — | — | — |
| PEG 400 | — | — | — | — | — |

Lubricant

| | | | | | |
|---|---|---|---|---|---|
| Magnesium stearate | — | — | — | — | — |
| Total [mg] | 30 | 20 | 20 | 20 | 20 |
| Dissolution at 30 min | 102 [99-105] | 97 [96-98] | 94 [92-95] | 92 [89-94] | 101 [100-102] |
| timepoint [%] (initial) | | | | | |
| Dissolution at 30 min | — | — | — | — | 99 [96-100] |
| timepoint [%] after 12 | | | | | |
| months at 25° C./60% rh | | | | | |
| open storage | | | | | |
| F rel [%] animal | 65 | — | — | — | — |
| AUC/D animal [kg*h/L] | 2.86 | — | — | — | — |
| Cmax/D animal [kg/L] | 0.610 | — | — | — | — |
| PXRD (initial) | amorphous | amorphous | amorphous | amorphous | amorphous |
| PXRD after 1 month at | — | amorphous | — | — | — |
| 40° C./75% rh open | | | | | |
| storage | | | | | |
| PXRD after 12 months at | — | — | — | — | amorphous |
| 25° C./60% rh open | | | | | |
| storage | | | | | |
| PXRD after 18 months at | amorphous | — | — | — | — |
| 25° C./60% rh closed | | | | | |
| storage | | | | | |
| PXRD after 24 months at | amorphous | — | — | — | — |
| 25° C./60% rh closed | | | | | |
| storage | | | | | |

| | 3-6 | 3-7 | 3-8 | 3-9 |
|---|---|---|---|---|
| Active ingredient (I), | — | — | — | — |
| crystalline modification I | | | | |
| Active ingredient (I), | 5.0 | 5.0 | 5.0 | 5.0 |
| amorphous form | | | | |

Solid dispersion base (polymer)

| | | | | |
|---|---|---|---|---|
| Polyvinylpyrrolidone (PVP) | — | — | — | 15.0 |
| HPMCAS MG | — | 10.0 | — | — |
| Kollidon VA64 | 10.0 | — | 15.0 | — |

Binder

| | | | | |
|---|---|---|---|---|
| Hypromellose 3 cP | — | — | — | — |

Solvent

| | | | | |
|---|---|---|---|---|
| Water | — | — | — | — |
| Ethanol 99% | 15.0 | — | 15.0 | 15.0 |
| Acetone | 15.0 | 70.0 | 15.0 | 15.0 |

Fillers

| | | | | |
|---|---|---|---|---|
| Microcrystalline cellulose | — | — | — | — |
| Lactose monohydrate | — | — | — | — |

Disintegration promoter

| | | | | |
|---|---|---|---|---|
| Croscarmellose sodium | 5.0 | 5.0 | 5.0 | 5.0 |

Surfactant

| | | | | |
|---|---|---|---|---|
| Sodium lauryl sulfate | — | — | — | — |
| PEG 4000 | — | — | — | — |
| PEG 6000 | — | — | — | — |
| PEG 400 | — | — | — | — |

Lubricant

| | | | | |
|---|---|---|---|---|
| Magnesium stearate | — | — | — | — |
| Total [mg] | 20 | 20 | 25 | 25 |
| Dissolution at 30 min | 100 [99-101] | 11 [9-14] | 100 [98-100] | 100 [98-101] |
| timepoint [%] (initial) | | | | |
| Dissolution at 30 min | 78 [70-84] | 25 [24-25] | 52 [49-56] | 88 [87-89] |
| timepoint [%] after 1 month | | | | |
| at 40° C./75% rh open storage | | | | |
| F rel [%] animal | — | — | — | — |
| AUC/D animal [kg*h/L] | — | — | — | — |
| Cmax/D animal [kg/L] | — | — | — | — |
| PXRD (initial) | amorphous | amorphous | amorphous | amorphous |
| PXRD after 1 month at | amorphous | amorphous | amorphous | amorphous |
| 40° C./75% rh open storage | | | | |
| PXRD after 18 months at | — | — | — | — |
| 25° C./60% rh closed storage | | | | |

### 6.3 Solid Pharmaceutical Dosage Forms (Tablets) Containing the Amorphous Solid Dispersion (ASD) Fluidized Bed Granulation Preparation Method (Table 3)

| | 4-1 | 4-2 | 4-3 | 4-4 | 4-5 | 4-6 | 4-7 |
|---|---|---|---|---|---|---|---|
| Active ingredient (I), | — | — | — | 50.0 | 50.0 | 50.0 | — |
| crystalline modification I | | | | | | | |
| Active ingredient (I), | 5.0 | 15.0 | 25.0 | — | — | — | 50.0 |
| amorphous form | | | | | | | |

Solid dispersion base (polymer)

| | | | | | | | |
|---|---|---|---|---|---|---|---|
| Polyvinylpyrrolidone (PVP) | 10.0 | 30.0 | 50.0 | 100.0 | 100.0 | 100.0 | 100.0 |
| HPMCAS MG | — | — | — | — | — | — | — |
| Kollidon VA64 | — | — | — | — | — | — | — |

Binder

| | | | | | | | |
|---|---|---|---|---|---|---|---|
| Hypromellose 3 cP | — | — | — | — | — | — | — |

Solvent

| | | | | | | | |
|---|---|---|---|---|---|---|---|
| Water | — | — | — | — | — | — | — |
| Ethanol 99% | 60.0 | 180.0 | 300.0 | 60.0 | 60.0 | 60.0 | 300.0 |
| Acetone | — | — | — | 240.0 | 240.0 | 240.0 | — |

Fillers

| | | | | | | | |
|---|---|---|---|---|---|---|---|
| Microcrystalline cellulose | 7.5 + 155 | 22.5 + 100 | 37.5 + 50.0 | 110.0 | 84.7 | 64.7 | — |
| Lactose monohydrate | 60.0 | 40.0 | 20.0 | — | — | — | — |

Disintegration promoter

| | | | | | | | |
|---|---|---|---|---|---|---|---|
| Croscarmellose sodium | 7.5 | 22.5 + 15.0 | 37.5 + 25.0 | 50.00 + 40.00 | 50.00 + 40.00 | 50.00 + 40.00 | 50.00 + 46.25 |

Surfactant

| | | | | | | | |
|---|---|---|---|---|---|---|---|
| Sodium lauryl sulfate | 2.5 | 2.5 | 2.5 | 3.5 | 3.5 | 3.5 | 2.5 |
| PEG 4000 | — | — | — | — | — | — | — |
| PEG 6000 | — | — | — | — | — | — | — |
| PEG 400 | — | — | — | — | — | — | — |

Lubricant

| | | | | | | | |
|---|---|---|---|---|---|---|---|
| Magnesium stearate | 2.5 | 2.5 | 2.5 | 1.8 | 1.8 | 1.8 | 1.25 |

Coating and film-forming agents and colorants/pigments

| | | | | | | | |
|---|---|---|---|---|---|---|---|
| Hypromellose 5 cP | — | — | — | — | — | — | — |
| Hypromellose 15 cP | 4.2 | 4.2 | 4.2 | — | — | — | — |
| Polyethylene glycol 3350 | 1.4 | 1.4 | 1.4 | — | — | — | — |
| Red iron oxide | 0.042 | 0.042 | 0.042 | — | — | — | — |
| Yellow iron oxide | — | — | — | — | — | — | — |
| Talc | — | — | — | — | — | — | — |
| Titanium dioxide | 1.358 | 1.358 | 1.358 | — | — | — | — |
| Total [mg] | 257 | 257 | 257 | 355.3 | 330 | 310 | 250 |
| Format (mm) | 12 × 6 | 12 × 6 | 12 × 6 | 14 × 7 | 14 × 7 | 14 × 7 | 12 × 6 |
| Disintegration time (min) | 5 | 4 | 8 | 10 | 9 | 10 | 14 |
| Dissolution at 30 min | 98 | 98 | 99 | 90 | 93 | 93 | 103 |
| timepoint [%] (initial) | [97-100] | [95-100] | [96-101] | [89-91] | [91-95] | [92-95] | [102-104] |
| Dissolution at 30 min | 101 | 100 | 105 | — | — | — | — |
| timepoint [%] after 30 | [97-107] | [94-104] | [102-107] | | | | |
| months at 25° C./60% rh | | | | | | | |
| closed storage | | | | | | | |
| PXRD (initial) | amorphous | amorphous | amorphous | — | amorphous | amorphous | amorphous |
| PXRD after 18 months at | amorphous | amorphous | amorphous | — | — | — | — |
| 25° C./60% rh closed storage | | | | | | | |
| PXRD after 30 months at | amorphous | amorphous | amorphous | — | — | — | — |
| 25° C./60% rh closed storage | | | | | | | |

| | 4-8 | 4-9 | 4-10 | 4-11 | 4-12 | 4-13 |
|---|---|---|---|---|---|---|
| Active ingredient (I), | — | — | — | — | — | — |
| crystalline modification I | | | | | | |
| Active ingredient (I) | 20.0 | 50.0 | 20.0 | 20.0 | 50.0 | 20.0 |
| amorphous form | | | | | | |

Solid dispersion base (polymer)

| | | | | | | |
|---|---|---|---|---|---|---|
| Polyvinylpyrrolidone (PVP) | 40.0 | 100.0 | 40.0 | 40.0 | 100.0 | 40.0 |
| HPMCAS MG | — | — | — | — | — | — |
| Kollidon VA64 | — | — | — | — | — | — |

Binder

| | | | | | | |
|---|---|---|---|---|---|---|
| Hypromellose 3 cP | — | — | — | — | — | — |

Solvent

| | | | | | | |
|---|---|---|---|---|---|---|
| Water | — | — | — | — | — | — |
| Ethanol 99% | 96.0 | 150.0 | 60.0 | 60.0 | 150.0 | 60.0 |
| Acetone | 24.0 | 150.0 | 60.0 | 60.0 | 150.0 | 60.0 |

Fillers

| | | | | | | |
|---|---|---|---|---|---|---|
| Microcrystalline cellulose | — | — | — | — | — | — |
| Lactose monohydrate | — | — | — | — | — | — |

Disintegration promoter

| | | | | | | |
|---|---|---|---|---|---|---|
| Croscarmellose sodium | 20.0 + 18.50 | 50.0 + 46.25 | 20.0 + 18.50 | 20.0 | 50.0 | 20.0 + 6.0 |

Surfactant

| | | | | | | |
|---|---|---|---|---|---|---|
| Sodium lauryl sulfate | 1.0 | 2.5 | 1.0 | — | — | — |
| PEG 4000 | — | — | — | — | — | — |
| PEG 6000 | — | — | — | — | — | — |
| PEG 400 | — | — | — | — | — | — |

Lubricant

| | | | | | | |
|---|---|---|---|---|---|---|
| Magnesium stearate | 0.50 | 1.25 | 0.50 | 0.40 | 1.00 | 0.40 |

Coating and film-forming agents and colorants/pigments

| | | | | | | |
|---|---|---|---|---|---|---|
| Hypromellose 5 cP | — | — | — | — | — | — |
| Hypromellose 15 cP | — | 3.00 | 1.80 | 1.80 | 3.00 | 1.56 |
| Polyethylene glycol 3350 | — | 1.00 | 0.60 | 0.60 | 1.00 | 0.52 |
| Red iron oxide | — | 0.03 | 0.018 | 0.018 | 0.03 | 0.0156 |
| Yellow iron oxide | — | — | — | — | — | — |
| Talc | — | — | — | — | — | — |
| Titanium dioxide | — | 0.97 | 0.582 | 0.582 | 0.97 | 0.5044 |
| Total [mg] | 100 | 255 | 103 | 83 | 206 | 89 |
| Format (mm) | 7 | 12 × 6 | 7 | 6 | 12 × 6 | 6 |
| Disintegration time (min) | 7 | 14 | 7 | 11 | 13 | 9 |
| Dissolution at 30 min | 100 | 100 | 100 | 102 | 100 | 98 |
| timepoint [%] (initial) | [100-101] | [98-101] | [100-101] | [101-103] | [99-102] | [96-100] |
| Dissolution at 30 min | — | — | — | — | — | 95 |
| timepoint [%]; after 9 | | | | | | [94-98] |
| months at 30° C./75% rh | | | | | | |
| closed storage | | | | | | |
| PXRD (initial) | amorphous | — | — | — | — | — |
| PXRD, after 1 month at | — | amorphous | amorphous | amorphous | — | — |
| 40° C./75% rh open storage | | | | | | |
| PXRD, after 9 months at | — | — | — | — | — | amorphous |
| 30° C./75% rh closed storage | | | | | | |

| | 4-14 | 4-15 | 4-16 | 4-17 | 4-18 |
|---|---|---|---|---|---|
| Active ingredient (I), | — | — | — | — | — |
| crystalline modification I | | | | | |
| Active ingredient (I) | 50.0 | 50.0 | 50.0 | 50.0 | 50.0 |
| amorphous form | | | | | |

Solid dispersion base (polymer)

| | | | | | |
|---|---|---|---|---|---|
| Polyvinylpyrrolidone (PVP) | 100.0 | — | — | — | 150.0 |
| HPMCAS MG | — | — | 100.0 | — | — |
| Kollidon VA64 | — | 100.0 | — | 150.0 | — |

Binder

| | | | | | |
|---|---|---|---|---|---|
| Hypromellose 3 cP | — | — | — | — | — |

Solvent

| | | | | | |
|---|---|---|---|---|---|
| Water | — | — | — | — | — |
| Ethanol 99% | 150.0 | 150.0 | — | 150.0 | 150.0 |
| Acetone | 150.0 | 150.0 | 700.0 | 150.0 | 1500 |

Fillers

| | | | | | |
|---|---|---|---|---|---|
| Microcrystalline cellulose | — | — | — | — | — |
| Lactose monohydrate | — | — | — | — | — |

Disintegration promoter

| | | | | | |
|---|---|---|---|---|---|
| Croscarmellose sodium | 50.0 + 15.0 | 5.0 + 15.0 | 5.0 + 15.0 | 5.0 + 15.0 | 5.0 + 15.0 |

Surfactant

| | | | | | |
|---|---|---|---|---|---|
| Sodium lauryl sulfate | — | — | — | — | — |
| PEG 4000 | — | — | — | — | — |
| PEG 6000 | — | — | — | — | — |
| PEG 400 | — | — | — | — | — |

Lubricant

| | | | | | |
|---|---|---|---|---|---|
| Magnesium stearate | 1.00 | 1.0 | 1.0 | 1.0 | 1.0 |

Coating and film-forming agents and colorants/pigments

| | | | | | |
|---|---|---|---|---|---|
| Hypromellose 5 cP | — | — | — | — | — |
| Hypromellose 15 cP | 2.4 | — | — | — | — |
| Polyethylene glycol 3350 | 0.8 | — | — | — | — |
| Red iron oxide | 0.024 | — | — | — | — |
| Yellow iron oxide | — | — | — | — | — |
| Talc | — | — | — | — | — |
| Titanium dioxide | 0.776 | — | — | — | — |
| Total [mg] | 220 | 216 | 216 | 266 | 266 |
| Format (mm) | 12 × 6 | 12 × 6 | 12 × 6 | 12 × 6 | 12 × 6 |
| Disintegration time (min) | 13 | 39 | 10 | >40 | 19 |
| Dissolution at 30 min | 99 | 10 | 64 | 12 | 91 |
| timepoint [%] (initial) | [98-100] | [9-12] | [58-69] | [12-14] | [88-94] |
| Dissolution at 30 min | — | 53 | 93 | 17 | 95 |
| timepoint [%] after 1 | | [52-55 | [90-98] | [17-18] | [91-96] |
| month at 40° C./75% rh | | | | | |
| open storage | | | | | |
| Dissolution at 30 min | 96 | — | — | — | — |
| timepoint [%] after 6 | [91-100] | | | | |
| months at 40° C./75% rh | | | | | |
| open storage | | | | | |
| Dissolution at 30 min | 100 | — | — | — | — |
| timepoint [%] after 9 | [98-102] | | | | |
| months at 30° C./75% rh | | | | | |
| closed storage | | | | | |
| PXRD (initial) | amorphous | amorphous | amorphous | amorphous | amorphous |
| PXRD, after 1 months at | — | — | — | — | — |
| 40° C./75% rh open storage | | | | | |
| PXRD, after 6 months at | amorphous | — | — | — | — |
| 40° C./75% rh open storage | | | | | |
| PXRD, after 9 months at | amorphous | — | — | — | — |
| 30° C./75% rh closed storage | | | | | |

### 6.4 Solid Pharmaceutical Dosage Forms (Grinded Extrudates) Containing the Amorphous Solid Dispersion (ASD) Hot Melt Extrusion Preparation Method (Comparison Examples) (Table 4)

| | 5-1 | 5-2 | 5-3 | 5-4 | 5-5 | 5-6 |
|---|---|---|---|---|---|---|
| Active ingredient (I), | — | — | — | 50.0 | 50.0 | 50.0 |
| crystalline modification I | | | | | | |
| Active ingredient (I) | 50.0 | 50.0 | 50.0 | — | — | — |
| amorphous form | | | | | | |

Solid dispersion base (polymer)

| | | | | | | |
|---|---|---|---|---|---|---|
| Polyvinylpyrrolidone (PVP) | 100.0 | 100.0 | — | 100.0 | 100.0 | — |
| HPMCAS MG | — | — | — | — | — | — |
| Kollidon VA64 | — | — | 100.0 | — | — | 100.0 |

Binder

| | | | | | | |
|---|---|---|---|---|---|---|
| Hypromellose 3 cP | — | — | — | — | — | — |

Solvent

| | | | | | | |
|---|---|---|---|---|---|---|
| Water | — | — | — | — | — | — |
| Ethanol 99% | — | — | — | — | — | — |
| Acetone | — | — | — | — | — | — |

Fillers

| | | | | | | |
|---|---|---|---|---|---|---|
| Microcrystalline cellulose | — | — | — | — | — | — |
| Lactose monohydrate | — | — | — | — | — | — |

Disintegration promoter

| | | | | | | |
|---|---|---|---|---|---|---|
| Croscarmellose sodium | — | — | — | — | — | — |

Surfactant

| | | | | | | |
|---|---|---|---|---|---|---|
| Sodium laurilsulfate | — | — | — | — | — | — |
| PEG 4000 | 11.1 | — | — | 11.1 | — | — |
| PEG 6000 | — | 11.1 | — | — | 11.1 | — |
| PEG 400 | — | — | — | — | — | — |

Lubricant

| | | | | | | |
|---|---|---|---|---|---|---|
| Magnesium stearate | — | — | — | — | — | — |
| Total [mg] | 161.1 | 161.1 | 150 | 161.1 | 161.1 | 150 |
| PXRD (initial) | amorphous | amorphous | amorphous | amorphous | amorphous | amorphous |

### 7. Bioavailability, Dissolution and Results

### 7.1 Comparison of Liquid Formulations and Amorphous Solid Dispersions (ASD) (in an Animal Model)

The liquid formulations of comparison examples 1-1 and 1-2 and the amorphous solid dispersion of example 3-1 have been tested in rats. Single doses were administered male rats (4 rats).

The liquid formulation was a suspension of the active ingredient (I) in crystalline modification I in an aqueous 0.5% tylose solution (Example 1-1).

The amorphous solid dispersion granulate of Example 3-1, was suspended in water prior to administration.

### Examples 1-1 and 3-1 have been Tested Against the PEG Solution of Example 1-2

Animals (male rats) were dosed with 3.00 mg active ingredient (I)/kg body weight. The volume of the application solution was 5.00 mL/kg body weight. Approximately 0.5 mL of whole blood were collected via an indwelling jugular catheter at 0, 0.25, 0.5, 0.75, 1, 2, 3, 5, 7, 24, 30, and 48 h post-dose. The blood samples were centrifuged in order to obtain plasma which was then transferred to the appropriately labeled vials and stored frozen (<−15° C.) until analysis. Plasma samples were analyzed via LC/MSMS for active ingredient (I) concentrations and pharmacokinetic parameters were calculated. The results are presented in Table 5.

TABLE 5
Comparison of the exposure of active ingredient
(I) in the rat for examples 1-1, 1-2 and 3-1

| | 1-1 | 1-2 | 3-1 |
|---|---|---|---|
| | Comparison | Comparison | Amorphous solid |
| | example | example | dispersion (ASD) |
| Dose [mg/kg] | 3.00 | 3.00 | 3.00 |
| AUC [kg*h/L] | 0.491 | 4.43 | 2.86 |
| cₘₐₓ [kg/L] | 0.089 | 0.794 | 0.610 |
| F rel [%] | 11 | 100 | 65 |

Result: The AUC and cₘₐₓ data reveal significant differences for the exposure of active ingredient (I) after oral application of the liquid formulations of examples 1-1 and 1-2 and the amorphous solid dispersion (ASD) of example 3-1. The PEG solution from Example 1-2 is used as a 100% reference standard. The exposure obtained after administration of the amorphous solid dispersion (ASD) of example 3-1 is significantly higher compared to the exposure of the liquid formulations of example 1-1. AUC is increased by a factor of approximately 5.8 and cₘₐₓ is increased by a factor of approximately 6.8 for the amorphous solid dispersion (ASD) of example 3-1.

The active ingredient (I) shows a low bioavailability and inferior dissolution profile when active ingredient (I) is used in crystalline modification I. The active ingredient (I) in its crystalline modification I shows a relative bioavailability of 11% only (example 1-1) whereas the amorphous solid dispersion comprising the active ingredient (I) in amorphous form shows a relative bioavailability of 65% (example 3-1) when administered in rats.

This proves that absorption and oral exposure are improved significantly by administering of active ingredient (I) in amorphous form solved in liquids (example 1-2) or as amorphous solid dispersion (ASD) of example 3-1 containing the active ingredient (I) in the form of an amorphous solid dispersion compared to liquid formulation of example 1-1, mimicking a standard IR tablet of example 2-1 containing the active ingredient (I) in crystalline modification I. The amorphous solid dispersion (ASD) provides a stabilized amorphous active ingredient (I), not showing the risk of crystallization compared to the standard IR tablet (example 2-1, comparison example).

### 7.2 Comparison of Standard IR Tablets with PEG Solution (in Humans)

Comparison examples 2-1, 2-2 and 2-3 are manufactured using fluidized bed granulation. In Comparison examples 2-2 and 2-3 active ingredient (I) in amorphous form is used to manufacture tablets with 50 mg (example 2-2) and 5 mg (example 2-3) dose. The tablet containing 5 mg of active ingredient (I) was tested as 25 mg dose (5 tablets of 5 mg active ingredient (I) each) against the PEG solution from example 1-2 in humans. The results are presented in Table 6.

TABLE 6
Comparison of the exposure of active ingredient
(I) in humans for examples 1-2 and 2-3

| Treatment | AUC/D (h/L) | Cₘₐₓ/D (1/L) |
|---|---|---|
| 5 × 5 mg IR tablet from example 2-3 | 0.237 | 0.0128 |
| 25 mg PEG solution from example 1-2 | 0.265 | 0.0149 |
| Ratio/rel. BA | 89.5% | 86.0% |

Result: Comparison example 2-3 showed a rel. BA of 89.5% for AUC/D and 86.0% for Cmax/D compared to the PEG solution from example 1-2.

If the same excipients and excipients concentrations are used but active ingredient (I) is used in form of crystalline modification T (comparison example 2-1) compared to the amorphous form in comparison examples 2-2 and 2-3 the dissolution rate decreases dramatically. The results are presented in Table 7.

TABLE 7
Comparison of the dissolution profiles
for examples 2-1 and 2-2 and 2-3

| Time Points [min] | 5 | 10 | 15 | 30 | 45 | 60 |
|---|---|---|---|---|---|---|
| example 2-3 [%] | — | — | 98.2 | 98.6 | 98.8 | 98.7 |
| example 2-2 [%] | — | — | 101.5 | 100.2 | 99.5 | 98.8 |
| example 2-1 [%] | 20.4 | 32.5 | 37.8 | 43.0 | 47.1 | 49.7 |

Result: The dissolution results of comparison example 2-1 are clearly lower compared to comparison examples 2-2 and 2-3, surrogating a decreased bioavailability (see FIG. 1). Comparison examples 2-2 and 2-3 contain active ingredient (I) in amorphous form without any stabilizing agent (e.g. polymers), having the risk of crystallization. If crystallization occurs, bioavailability will be decreased.

### 7.3 Comparison of Amorphous Solid Dispersions (ASD) with Liquid Formulations (in an Animal Model)

The amorphous solid dispersion (ASD) from examples 3-1 to 3-5 are manufactured via fluidized bed granulation. The used polymer serves as a stabilizer to prevent crystallization. The active ingredient (I) in crystalline modification I used in example 3-3 is transferred to the amorphous state by using this manufacturing process. It is possible to use a mixture of filler and disintegration promoter as carrier (example 3-1) or only disintegration promoter (examples 3-2 to 3-5). The solvent can be either ethanol (example 3-1 and example 3-2) or mixtures of ethanol and acetone (examples 3-3 to 3-5).

The exposure of active ingredient (I) from amorphous solid dispersion (ASD) (example 3-1) compared to liquid formulations (examples 1-1 and 1-2) is shown in Table 5.

### 7.4 Comparison of Pharmaceutical Dosage Forms of Amorphous Solid Dispersions (ASD) with Standard IR Tablets (in Humans)

Examples 4-1 to 4-14 are describing pharmaceutical dosage forms (tablets) comprising amorphous solid dispersion (ASD) using granulates manufactured in examples 3-1 to 3-5. For the examples 4-1 to 4-3 the granulate of example 3-1 is formulated into tablets with varying amounts of active ingredient (I) and varying amounts of additional excipients.

TABLE 8
Comparison of the dissolution profiles of
examples 4-1 to 4-3 and 4-14 to comparison

| Time Points [min] | 0 | 15 | 30 | 45 | 60 |
|---|---|---|---|---|---|
| example 4-1 [%] | — | 89.6 | 98.5 | 99.4 | 99.1 |
| example 4-2 [%] | — | 96.0 | 98.2 | 96.7 | 96.7 |
| example 4-3 [%] | — | 91.0 | 99.3 | 99.0 | 98.4 |
| example 4-14 [%] | — | 73 | 99 | 99 | 99 |
| example 2-2 [%] | — | 101.5 | 100.2 | 99.5 | 98.8 |

Result: For all examples (examples 4-1 to 4-3 and 4-14) the dissolution profiles are comparable to the dissolution profile of comparison example 2-2.

Example 4-3 describes the pharmaceutical dosage forms (tablets) comprising amorphous solid dispersion (ASD) using granulates manufactured in examples 3-1. The tablet containing 25 mg of active ingredient (I) was tested as 25 mg dose against a tablet with 50 mg dose from example 2-2 in humans. The results are presented in Table 9.

TABLE 9
Comparison of the exposure of active ingredient
(I) in humans for examples 4-3 and 2-2

| Treatment | AUC/D (h/L) | Cₘₐₓ/D (1/L) |
|---|---|---|
| 25 mg ASD tablet from example 4-3 | 0.253 | 0.0134 |
| 50 mg IR tablet from example 2-2 | 0.268 | 0.0141 |
| Ratio/rel. BA | 94.3% | 95.5% |

Example 4-14 describes the pharmaceutical dosage forms (tablets) comprising amorphous solid dispersion (ASD) using granulates manufactured in examples 3-5. The tablet containing 50 mg of active ingredient (I) was tested as 50 mg dose against 25 mg dose from example 4-3 in humans. The results are presented in Table 9a.

TABLE 9a
Comparison of the exposure of active ingredient
(I) in humans for examples 4-3 and 4-14

| Treatment | AUC/D (h/L) | Cₘₐₓ/D (1/L) |
|---|---|---|
| 25 mg ASD tablet from example 4-3 | 0.268 | 0.0141 |
| 50 mg ASD tablet from example 4-14 | 0.254 | 0.0126 |
| Ratio/rel. BA | 94.9% | 88.8% |

Result: Tablets (example 4-3) manufactured by using the amorphous solid dispersion (ASD) from example 3-1 showed high relative bioavailability in humans (rel. BA of 94.3% for AUC/D and 95.5% for Cmax/D) compared to the IR tablet of example 2-2. In addition, tablets (example 4-14) manufactured by using the amorphous solid dispersion (ASD) from example 3-5 showed high relative bioavailability in humans (rel. BA of 94.9% for AUC/D and 88.8% for Cmax/D) compared to the tablets from example 4-3. It could be concluded that the manufacturing technique of fluidized bed granulation leads to high bioavailability, since it transfers the active ingredient (I) to an amorphous form independently of the initial modification of active ingredient (I) (amorphous form or crystalline modification I) used for the process and at the same time stabilizes the active ingredient (I) in the amorphous form to prevent crystallization.

That by using a fluidized bed granulation and excipients according to the present invention, the major influence of the modification of the active ingredient (I) (amorphous form or crystalline modification I) on the bioavailability in humans is levelled out, since the initial polymorphic form used for the manufacturing of the ASD will be transferred during the manufacturing process to a polymeric-stabilized amorphous form, is proven by examples 4-4 to 4-6. In these examples 4-4 to 4-6 the amorphous solid dispersion (ASD) from example 3-3 is used to manufacture tablets comprising different amounts of filler in the tablet. It could be concluded that the amount of filler is not critical for a fast dissolution of the active ingredient (I) and all examples 4-4 to 4-6 let to tablets with the desired dissolution characteristics. Even the reduction of the filler to zero in example 4-7 (using the amorphous solid dispersion (ASD) of example 3-2) was possible for a tablet formulation having a dose of 50 mg with the desired dissolution characteristics. In example 4-8 the amorphous solid dispersion (ASD) of example 3-4 was used to prove, that the elimination of filler in the postblend is also possible to manufacture a tablet of 20 mg with the desired dissolution characteristics. For examples 4-9 to 4-14 the granulate of example 3-5 has been used to manufacture tablets comprising different levels/amount of disintegration promoter. It could be shown that it was possible to use a wide range down to zero of disintegration promoter in the postblending step, while achieving at the same time the most desired dissolution properties of the active ingredient (I) from the tablet. For examples 4-11 to 4-14 it could be shown, that it was also possible to achieve the desired dissolution properties of active ingredient (I) from the tablet without the use of sodium lauryl sulfate as a surfactant. The comparison of the dissolution rate of active ingredient (I) tablets of examples 4-1 to 4-14 are mentioned in Table 3.

Comparison of Various Polymers as Solid Dispersion Base:

For examples 4-15 and 4-17 Kollidon VA64 and for example 4-16 HPMCAS MG have been used as solid dispersion base. Surprisingly, the resulting dissolution values remain very low (for example 4-15: 11%, for example 4-16: 65%, for example 4-17: 13%) and fail to meet the release of active ingredient (I) of at least 85% after 30 minutes criteria. In addition, especially the Kollidon VA 64 tablets showed prolonged disintegration times of more than 30 minutes. It could be concluded that neither HPMCAS MG or Kollidon VA64 are suitable to achieve the desired dissolution criteria of a release of active ingredient (I) of at least 85% after 30 minutes. This clearly shows the superiority of PVP selected as a solid solution base.

In example 4-18 the granulate of example of 3-9 was used to manufacture tablets that contain a ratio of active ingredient (I) to solid dispersion base of 1 to 3. The example 4-18 meets the dissolution criteria of a release of active ingredient (I) of at least 85% after 30 minutes, but reveals a nearly 1.5 fold prolonged disintegration time and a reduced drug-load compared to example 4-14.

### 7.5 Hot-Melt Extrusion to Manufacture Pharmaceutical Dosage Forms Containing the Active Ingredient (I)—Influence on Enantiomeric Purity

The hot-melt extrusion process generated high amounts of the wrong enantiomer, which is known, that it is inactive in humans, independent of the used solid dispersion base, the surfactant or the modification of the active ingredient (I) (amorphous form or crystalline modification I). The results are presented in Table 10.

TABLE 10
Influence of hot-melt extrusion and fluidized bed granulation
on enantiomeric purity of active ingredient (I)

| Granulate | Amount of wrong | Amount of wrong |
|---|---|---|
| according | enantiomer in starting | enantiomer in the |
| to example | active ingredient (I) | granulate |
| 5-1 | <0.2% | 4.5% |
| 5-2 | <0.2% | 3.6% |
| 5-3 | <0.2% | 5.8% |
| 5-4 | 1.2% | 8.8% |
| 5-5 | 1.2% | 8.5% |
| 5-6 | 1.2% | 5.9% |
| 3-1 | <0.2% | <0.2% |
| 3-4 | <0.2% | <0.2% |
| 3-5 | <0.2% | <0.2% |

Result: For the hot melt extrusion process (examples 5-1 to 5-6 from Table 10) generation of the wrong enantiomer, which is ineffective in-vivo, is clearly increased/escalated compared to the initial enantiomerically purity of the active ingredient (I). Therefore hot-melt extrusion is not considered to be an appropriate process. In contrast to the hot-melt extrusion process a fluidized bed granulation process does not lead to an increased building of the wrong enantiomer of active ingredient (I) (examples 3-1, 3-4 and 3-5 from Table 10).

This shows that active ingredient (I) has to be present in amorphous form, that crystallization of active ingredient (I) has to be prevented and enantiomeric purity has to be safeguarded during manufacturing and storage of the tablets (pharmaceutical dosage form) comprising active ingredient (I).

These data support that an amorphous solid dispersion (ASD) comprising amorphous active ingredient (I) stabilized by polyvinylpyrrolidone (PVP) and croscarmellose sodium and manufactured by a fluidized bed granulation shows a superior dissolution behaviour characterized by a release of active ingredient (I) of at least 85% after 30 minutes. Furthermore, the enantiomeric purity is safeguarded, the amorphous solid dispersion comprising active ingredient (I) shows (long-term) stability by open storage at 40° C. and 75% relative humidity (harsh conditions) and the bioavailability is good even for tablets (pharmaceutical dosage form) which have been stored at these harsh conditions. Results are shown in Table 3.

### 8. Disintegration Method

According to the European Pharmacopoeia, 10th Edition, last revision of monograph 01/2020, the oral solid dosage form is tested in a rigid basket-rack apparatus. The six test specimens are placed individually in a tube of the basket and a disk is added. The apparatus is operated by using water as a medium at 37+/−2° C. Results are shown in Table 1 and Table 3.

### 9. PXRD Method

Powder x-ray diffraction (PXRD) data were recorded on a STOE STADI P diffractometer using monochromatized Cu-K alpha 1 radiation, a position sensitive detector, at generator settings of 40 kV and 40 mA. The samples were collected in transition mode and prepared as a thin layer between two foils. The scanning rage was between 10° and 26° 2 theta with a 0.1° step at 60 seconds/step.

### 10. Solubility

The solubility of the active ingredient (I) in crystalline form was determined in different solvents. The results are presented in Table 11.

TABLE 11
Solubility of active ingredient (I) in
crystalline form in different solvents

| Amount Ethanol [%] | Amount Acetone [%] | Solubility* [μg/mL] |
|---|---|---|
| 100 | 0 | 35000 |
| 50 | 50 | 180000 |
| 0 | 100 | 80000 |

*solubility may vary slightly among different batches

### 11. Explanation of the Figures

FIG. 1: Comparison of dissolution profile for examples 2-1, 2-2 and 2-3 revealing the influence of usage of active ingredient (I) in the crystalline modification I.

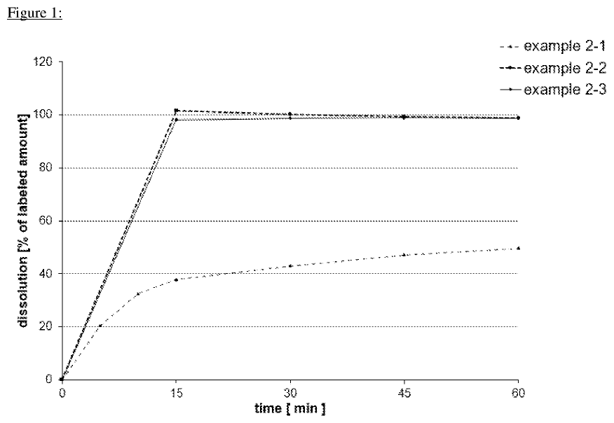

1. A solid pharmaceutical dosage form for oral administration comprising an amorphous solid dispersion (ASD), containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzena-heptaphane-7⁴-carboxamide (active ingredient (I)) in a pharmaceutically acceptable matrix.

2. The solid pharmaceutical dosage forms for oral administration according to claim 1, comprising
a) an amorphous solid dispersion (ASD), containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix,
b) at least one lubricant,
c) at least one disintegration promoter,
d) optionally one or more fillers, and
e) optionally one or more surfactants.

3. The solid pharmaceutical dosage form for oral administration according to claim 1, characterized in that at least 85% of active ingredient (I) are released into the release medium after 30 minutes, according to the release method of the European Pharmacopoeia using apparatus 2 (paddle).

4. The solid pharmaceutical dosage form for oral administration according to claim 1, characterized in that the dosage form is a tablet.

5. The solid pharmaceutical dosage form for oral administration according to claim 1, characterized in that the pharmaceutically acceptable matrix consists of the combination of the solid dispersion base and the carrier.

6. The solid pharmaceutical dosage form for oral administration according to claim 5, characterized in that the solid dispersion base is the polymer polyvinylpyrrolidone (PVP).

7. An amorphous solid dispersion (ASD) comprising (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix.

8. The amorphous solid dispersion (ASD) according to claim 7, comprising (4S)-2⁴-chloro-4-ethyl-7³-fluoro-3⁵-methoxy-3²,5-dioxo-14-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) in a pharmaceutically acceptable matrix and optionally sweeteners, flavoring agents and colorants.

9. The amorphous solid dispersion (ASD) according to claim 7, characterized in that (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) is present in amorphous form.

10. The amorphous solid dispersion (ASD) according to claim 7, characterized in that the pharmaceutically acceptable matrix consists of the combination of the solid dispersion base and the carrier.

11. The amorphous solid dispersion (ASD) according to claim 10, characterized in that the solid dispersion base is the polymer polyvinylpyrrolidone (PVP).

12. The amorphous solid dispersion (ASD) according to claim 10, characterized in that the carrier is selected from the groups of fillers, lubricants, disintegration promoters, surfactants, sweeteners, flavoring agents and/or colorants or a combination thereof.

13. A method for preparing an amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)), characterized in that the amorphous solid dispersion (ASD) is prepared by wet granulation.

14. The method according to claim 13, characterized in that the amorphous solid dispersion (ASD) is prepared by fluidized bed granulation, in which the active ingredient (I) is solved in the granulating fluid, which contains a mixture of 50% ethanol and 50% acetone, and is introduced into the fluidized bed granulator.

15. The amorphous solid dispersion (ASD) containing (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide (active ingredient (I)) producible by the method according to claim 13.
